(19) United States
(12) Patent Application Publication
(10) Pub. No.: US 2023/0023440 A1
(43) Pub. Date: Jan. 26, 2023

(54) SYSTEMS FOR PREDICTING INTRAOPERATIVE PATIENT MOBILITY AND IDENTIFYING MOBILITY-RELATED SURGICAL STEPS

(71) Applicant: Carlsmed, Inc., Carlsbad, CA (US)

(72) Inventors: Niall Patrick Casey, Carlsbad, CA (US); Justin Esterberg, Mercer Island, WA (US)

(21) Appl. No.: 17/868,729

(22) Filed: Jul. 19, 2022

Related U.S. Application Data
(60) Provisional application No. 63/223,827, filed on Jul. 20, 2021.

(51) Int. Cl. A61B 34/10; A61B 34/00

(52) U.S. Cl. CPC A61B 34/10; A61B 34/25; A61B 2034/105; A61B 2034/107; A61B 2034/252; A61B 2034/104

(57) ABSTRACT

Computer-implemented methods for modeling a surgical correction for a patient, and associated systems are disclosed herein. In some embodiments, the method includes obtaining patient data. The image data can depict a native anatomical configuration of a region of a patient's spine. The method also includes generating a virtual model of the patient's spine in the native anatomical configuration and/or a corrected anatomical configuration. The method can also include identifying one or more soft tissue surgical steps, predicting an effect of the soft tissue surgical steps, and generating a surgical plan for achieving the corrected anatomical configuration. The soft tissue surgical step can adjust an intraoperative mobility of vertebrae of the spine to achieve the corrected anatomical configuration. The surgical plan includes at least one of the soft tissue surgical steps to help facilitate movement of the vertebrae to the corrected anatomical configuration.

### CROSS-REFERENCE TO RELATED APPLICATION

The present application claims priority to U.S. Provisional Patent Application No. 63/223,827, filed Jul. 20, 2021, the disclosure of which is incorporated by reference herein in its entirety.

### TECHNICAL FIELD

The present disclosure is generally related to patient-specific medical care, including systems using predictive analytics to predict intraoperative mobility, such as distraction and lordosis, and to identify new and/or additional surgical steps to improve medical procedures.

### BACKGROUND

Orthopedic surgeries can correct, or reduce, numerous different maladies in a variety of contexts, including spine surgery, hand surgery, shoulder and elbow surgery, total joint reconstruction (arthroplasty), skull reconstruction, pediatric orthopedics, foot and ankle surgery, musculoskeletal oncology, surgical sports medicine, and orthopedic trauma. Spine surgery itself may encompass a variety of procedures and targets, such as one or more of the cervical spine, thoracic spine, lumbar spine, or sacrum, and may be performed to treat a deformity or degeneration of the spine and/or related back pain, leg pain, or other body pain. Common spinal deformities that may be treated using an orthopedic implant include irregular spinal curvature such as scoliosis, lordosis, or kyphosis (hyper- or hypo-), and irregular spinal displacement (e.g., spondylolisthesis). Other spinal disorders that can be treated using an orthopedic implant include osteoarthritis, lumbar degenerative disc disease or cervical degenerative disc disease, lumbar spinal stenosis, and cervical spinal stenosis. The success of orthopedic surgeries is often dependent on a resulting anatomical configuration, which is in turn often dependent on intraoperative mobility of the patient's body and/or the surgeon's instruments.

### BRIEF DESCRIPTION OF THE DRAWINGS

FIG. 1 is a network connection diagram illustrating a computing system for providing patient-specific medical care according to embodiments of the present technology.

FIG. 2 illustrates a computing device suitable for use in connection with a system of the type illustrated in FIG. 1.

FIG. 3 is a flow diagram illustrating a method for providing patient-specific medical care, according to some embodiments of the present technology.

FIGS. 4A-4C illustrate various examples of a virtual model of a patient's native anatomical configuration in accordance with some embodiments of the present technology.

FIGS. 5A-5C illustrate exemplary data sets that may be used and/or generated in connection with the methods in accordance with some embodiments of the present technology.

FIG. 6 is a flow diagram illustrating a pre-operative method for generating a patient-specific plan for a surgical procedure in accordance with some embodiments of the present technology.

FIG. 7 is a flow diagram illustrating an intraoperative method for adapting a patient-specific plan for a surgical procedure to patient-specific anatomical structures during the surgical procedure in accordance with some embodiments of the present technology.

FIG. 8 is a flow diagram illustrating an intraoperative method for adapting a patient-specific plan for a surgical procedure to patient-specific anatomical structures during the surgical procedure in accordance with further embodiments of the present technology.

FIG. 9 illustrates an exemplary surgical plan with stages for adjusting intraoperative mobility, according to an embodiment.

The drawings have not necessarily been drawn to scale. Similarly, some components and/or operations can be separated into different blocks or combined into a single block for the purpose of discussion of some of the implementations of the present technology. Moreover, while the technology is amenable to various modifications and alternative forms, specific implementations have been shown by way of example in the drawings and are described in detail below. The intention, however, is not to limit the technology to the particular implementations described.

### DETAILED DESCRIPTION

### Overview

Systems and methods for modeling and/or updating a plan for a medical treatment for a patient are disclosed herein. An example application of the systems and methods can be applied to a method for generating and/or updating a plan for a spinal surgical procedure. In some embodiments, the method can include obtaining patient data and generating a virtual model of the patient's spine in a corrected anatomical configuration based on the patient data. In some embodiments, the patient data includes image data of one or more regions of a patient's spine that includes a depiction of a native anatomical configuration of the patient's spine. The method can then include identifying one or more ancillary, alternative, additional, and/or unconventional steps and/or procedures (referred to collectively as “additional steps” or “ancillary steps”) for adjusting intraoperative mobility of vertebrae of the patient's spine to achieve the corrected anatomical configuration. The additional steps can include manipulating soft tissue surrounding the patient's spine (e.g., ligaments, muscles, nerves, discs, and the like) and/or additional vertebrae manipulation (e.g., vertebrae outside of the target vertebrae and/or additional surgical steps to the target vertebrae). Specific examples of the additional steps can include severing a ligament along the subject's spine; removing at least a portion of an annulus of intervertebral disc; resecting cartilage along the spine; performing an additional decompression procedure, an osteotomy, and/or facetectomy; interrupting an unintended (or undesired) bone fusion; and/or addressing malformities and/or irregularities in a bone (e.g., addressing fibrous dysplasia). Next, the method can include generating a surgical plan that includes at least one of the additional surgical steps.

In some embodiments, a predictive modeling system for orthopedic corrections can predict patient mobility, such as distraction and/or lordosis of the spine. In pre-operative planning, the system can predict interoperative mobility to help a physician understand how anatomical features can be moved during a surgical procedure. In some embodiments, the system can determine predictions based on, for example, soft tissue release, boney tissue release, tissue characteristics (e.g. bone quality, bone density, tissue density distribution, bone strength, etc.), and/or joint characteristics (e.g. joint stiffness, joint mobility, etc.). The surgeon's surgical techniques can affect the interoperative characteristics of the spine, so the surgeon's technique history can be incorporated into predictions. The soft tissue release can include, with limitation, release of ligaments, annulus, cartilage, or the like. Bony tissue release can include, with limitation, osteotomy, interruption of undesirable fusion, facetectomy, malformed bones, irregularities of bone, or the like. Soft tissue release and/or bone tissue release can be predicted to generate additional predictions (e.g., intra-operative predictions, post-operative predictions, etc.).

The system can predict post-operative corrections based on, for example, local anatomical environment conditions. Image analysis can be used to determine actual mobility, pre-operative mobility, post-operative mobility (e.g. mobility after surgical intervention). Predictive models can be incorporated into surgical robotic environments. The predictive modeling can be incorporated into algorithms or software configured to perform virtual surgeries based on, for example, surgical plans, local anatomy, and/or expected surgical interventions. In some embodiments, predictive modelling can be incorporated into surgical plans to provide comprehensive predictions.

The system can be identified potential surgical maneuvers to navigate anatomy. For example, the system can identify, without limitations, bridging osteophytes, auto-fused segments, and/or other anatomical features that affect mobilities. The system can perform virtual corrections to virtually cut through such features. In some procedures, the system can virtually cut a bridging osteophyte and can notify the surgeon of the virtual when viewing patient images, simulations, etc. The system can segment one or more mobility limiting features pre-operatively to provide surgical planning. Surgical paths can be determined based on virtual cuts for desired interoperative mobility of the spine. The system can determine optimal positions for performing surgical steps that enable desired intraoperative mobility to facilitate insertion of implants, repositioning adjacent anatomical elements (e.g. adjacent vertebral bodies, adjacent spinous processes, or the like), etc.

In some embodiments, systems can predict amounts of correction on a multi-level or per level basis for each patient. The system can plan one or more surgical stages of future cases. Level-by-level machine learning algorithms can be used to predict corrections for levels. Records of post-operative patient data (e.g., data for each level) can be collected to determine surgical steps that facilitated achievement of targeted mobility, spinal correction, etc. In some embodiments, corrections on a per level basis can be determined, at least in part, via image analysis.

The system can generate one or more surgical plans, patient-specific implants, or the like. After completion of surgical procedure, patient data can be retrieved. The retrieved patient data can include post-operative measurements, imaging (e.g., imaging captured over a period of time) for evaluating corrections, disease progression scores, or the like. Data can be collected for specific regions, such as anterior side of the spine, posterior side of the spine, etc., and can be tagged for each case. The system can determine optimal surgical steps for providing mobility for facilitating surgical interventions while also providing targeted post-operative corrections. In some fusion procedures, a posterior facet capsule can be removed and burring of the joint be performed for moving anatomical features to target fusions positions.

The system can identify alterations to surgical plans to predict interoperative ability of the patient's spine. Features or obstacles that affect the interoperative spine mobility can be identified for the physician. The procedures for adjusting the mobility of anatomical elements can be ancillary spine procedures performed prior to surgical steps for providing permanent spinal correction. Multiple simulations can be performed to predict how ancillary spine procedures will affect the targeted outcome. Additionally, ancillary surgical procedures can include steps for reversing the intraoperative mobility increase and/or inhibiting or limiting post-operative mobility. For example, if ligaments along the spine are severed to intra-operatively distract adjacent vertebra, the ligaments can be coupled back together after, for example, implanting an intervertebral device. This allows for increased mobility intra-operatively while restoring normal function. Ancillary spinal procedures can also include surgical steps for increasing mobility permanently by, for example, cutting undesirable fusion between bone tissue, removing bone tissue (e.g. burring of joint interfaces), or the like. Accordingly, ancillary spinal procedures can temporarily or permanently adjust mobility of individual joints, a group of joints, targeted body part, etc.

In various embodiments, the method includes receiving intraoperative updates to the patient data; comparing the patient data to one or more reference patient cases; identifying one or more additional steps based on the intraoperative patient data; identifying one or more obstacles to the surgical procedure and/or the desired anatomical configuration; predicting an anatomical configuration resulting from a surgical procedure including the additional steps; receiving intraoperative input from a surgeon on the obstacles and/or additional steps; and/or generating an updated plan for the surgical procedure. In various embodiments, the virtual modeling, plan generation, identification of obstacles, identification of additional steps, and/or prediction of anatomical outcomes is performed by a machine learning model, artificial intelligence (AI) model, neural network and/or any other suitable computer modeling module.

The method can also include simulating the intraoperative mobility using the virtual model. In such embodiments, the virtual model allows a healthcare provider (e.g., a surgeon) and/or the patient to visualize the intraoperative mobility associated with the additional steps. For example, the virtual model can identify the intraoperative mobility attributable to the one or more additional surgical steps, as well as the changes after each of the steps. In some embodiments, the virtual model also allows the healthcare provider to virtually simulate the surgical steps in the updated plan. In some embodiments, the method then includes receiving inputs from the healthcare provider that can then be used in generating the surgical plan. For example, the inputs can include one or more selections of the identified additional steps to be incorporated into the surgical plan.

In some embodiments, the method includes predicting intraoperative spinal mobility based on the one or more additional surgical steps incorporated into the updated plan and determining one or more intraoperative correction values based on the predicted mobility. The intraoperative correction values can include at least one of a maximum distraction, lordosis correction, kyphosis correction, scoliosis correction, and spondylolisthesis correction. In some embodiments, the method includes predicting post-operative spinal mobility based on the one or more soft tissue surgical steps being performed.

In some embodiments, the method can include generating plans for a plurality of surgical procedures, predicting an anatomical outcome from each of the plurality of surgical procedures, receiving selection of one of the plurality of surgical procedures, and generating a full surgical plan based on the selected plan. In some embodiments, for example, the plurality of surgical procedures includes a laminectomy, a laminotomy, a microdiscectomy, a foraminotomy, and/or an osteophyte procedure, allowing a healthcare provider to select from a range of spinal decompression procedures.

For ease of understanding, the systems and methods disclosed herein are primarily discussed in the context of examples of spinal surgeries and/or treatments. However, one of skill in the art will understand that the scope of the invention is not so limited. For example, the systems and methods disclosed herein can also be applied to various other medical applications, such as various other orthopedic surgeries and various other anatomical structures in living organisms.

### DESCRIPTION OF THE FIGURES

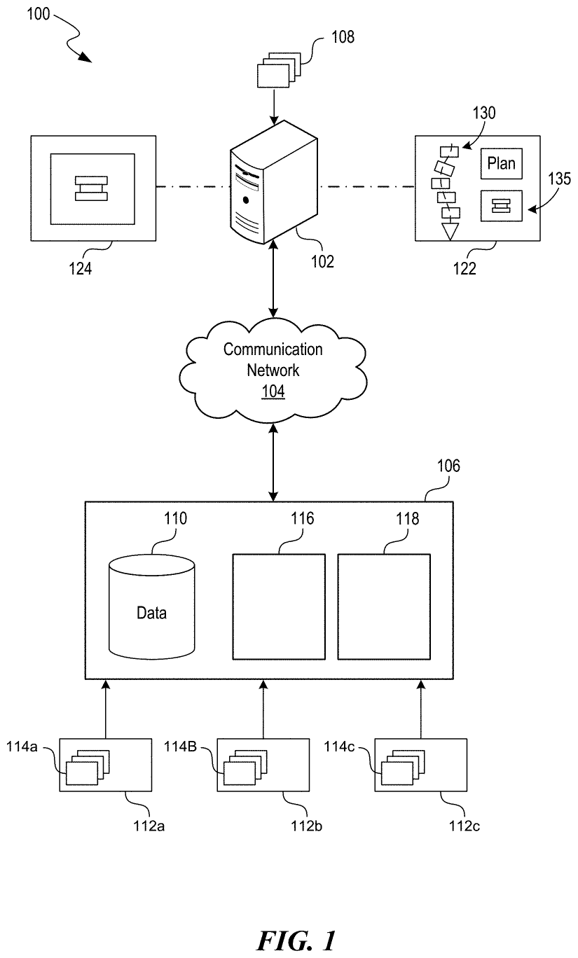

FIG. 1 is a network connection diagram illustrating a computing system 100 for providing patient-specific, predictive recommendations for medical care, according to some embodiments of the present technology. As described in further detail herein, the system 100 is configured to generate and/or update a medical treatment plan for a patient while identifying one or more ancillary, alternative, additional, and/or unconventional steps (referred to collectively as “additional steps” or “ancillary steps”) for the treatment plan. The system 100 is also configured to predict a result or outcome of the treatment plan (e.g., an exact anatomical correction provided to a patient's spine, a mobility of the patient's spine, or any other suitable result). In some embodiments, the system is configured to both identify the one or more additional steps and accurately predict a result of each identified step alone and in combination.

In some embodiments, the system 100 is configured to generate a medical treatment plan for a patient suffering from an orthopedic or spinal disease or disorder, such as trauma (e.g., fractures), cancer, deformity, degeneration, pain (e.g., back pain, leg pain), irregular spinal curvature (e.g., scoliosis, lordosis, kyphosis), irregular spinal displacement (e.g., spondylolisthesis, lateral displacement axial displacement), osteoarthritis, lumbar degenerative disc disease, cervical degenerative disc disease, lumbar spinal stenosis, or cervical spinal stenosis, or a combination thereof. The medical treatment plan can include surgical information, surgical plans, technology recommendations (e.g., device and/or instrument recommendations), and/or medical device designs. For example, the medical treatment plan can include at least one treatment procedure (e.g., a surgical procedure or intervention) and/or at least one design for a medical device (e.g., an implanted medical device (also referred to herein as an “implant” or “implanted device”) or implant delivery instrument).

In some embodiments, the system 100 generates and/or updates a medical treatment plan with steps of a procedure that are customized for a particular patient or group of patients, also referred to herein as a “patient-specific” or “personalized” treatment plan. The patient-specific treatment plan can include at least one patient-specific surgical procedure, at least one patient-specific (additional) step of the surgical procedure and/or at least one design for a patient-specific medical device that is designed and/or optimized for the patient's particular characteristics (e.g., condition, anatomy, soft tissue features, pathology, condition, medical history), a surgeon's preferred operations, and/or other steps in the surgical procedure. For example, the patient-specific surgical procedure can include steps predicted to increase intraoperative mobility within the patient's specific anatomy, thereby improving post-operative results. However, it shall be appreciated that a patient-specific treatment plan can also include aspects that are not customized for the particular patient. For example, a patient-specific or personalized surgical procedure can include one or more instructions, portions, steps, etc. that are non-patient-specific. Likewise, a patient-specific or personalized design for a medical device can include one or more elements that are non-patient-specific. Personalized implant designs can be used to manufacture or select patient-specific technologies, including medical devices, instruments, and/or surgical kits. For example, a personalized surgical kit can include one or more patient-specific devices, patient-specific instruments, non-patient-specific technology (e.g., standard instruments, devices, etc.), instructions for use, patient-specific treatment plan information, or a combination thereof.

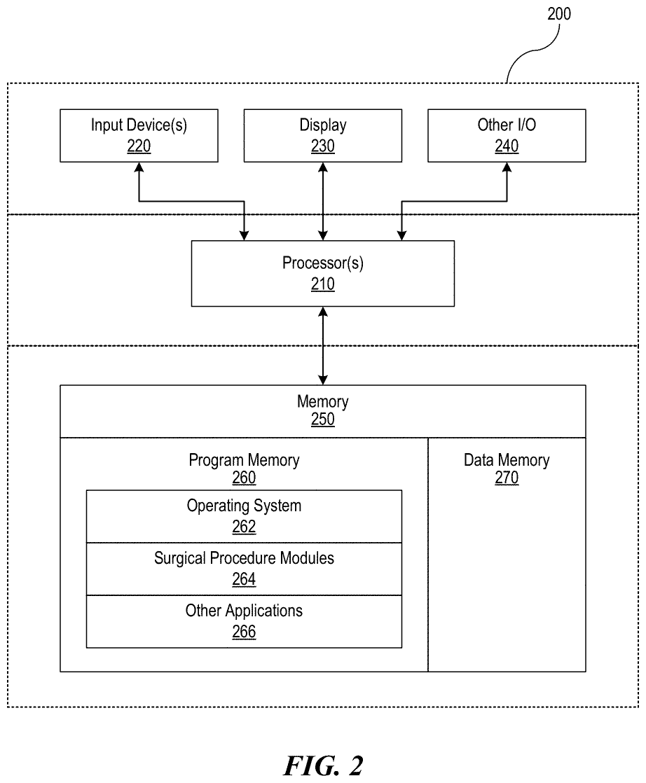

As illustrated in FIG. 1, the system 100 includes a client computing device 102, which can be a user device, such as a smart phone, mobile device, laptop, desktop, personal computer, tablet, phablet, or other such devices known in the art. As discussed further herein, the client computing device 102 can include one or more processors, and memory storing instructions executable by the one or more processors to perform the methods described herein. The client computing device 102 can be associated with a healthcare provider that is treating the patient. Although FIG. 2 illustrates a single client computing device 102, in alternative embodiments, the client computing device 102 can instead be implemented as a client computing system encompassing a plurality of computing devices, such that the operations described herein with respect to the client computing device 102 can instead be performed by the computing system and/or the plurality of computing devices.

The client computing device 102 is configured to receive a patient data set 108 associated with a patient to be treated. The patient data set 108 can include data representative of the patient's condition, anatomy, pathology, medical history, preferences, and/or any other information or parameters relevant to the patient. For example, the patient data set 108 can include medical history, surgical intervention data, treatment outcome data, progress data (e.g., physician notes), patient feedback (e.g., feedback acquired using quality of life questionnaires, surveys), clinical data, provider information (e.g., physician, hospital, surgical team), patient information (e.g., demographics, sex, age, height, weight, type of pathology, occupation, activity level, tissue information, health rating, comorbidities, health related quality of life (HRQL)), vital signs, diagnostic results, medication information, allergies, image data (e.g., camera images, Magnetic Resonance Imaging (MRI) images, ultrasound images, Computerized Aided Tomography (CAT) scan images, Positron Emission Tomography (PET) images, X-Ray images), diagnostic equipment information (e.g., manufacturer, model number, specifications, user-selected settings/configurations, etc.), or the like. In some embodiments, the patient data set 108 includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine. In some embodiments, the patient data set 108 includes data representing the patient's soft tissue features. In some embodiments, the data representing the patient's soft tissue features include data on the size, health, strength, flexibility, growth, and/or integration of ligaments in the patient's anatomy. For example, for a portion of the patient's spine, the data representing the patient's soft tissue features can include measurements of the ligamentum flavum, anterior longitudinal ligament, posterior longitudinal ligament, interspinous ligament, supraspinous ligament, intertransverse ligament, facet capsular ligament, and/or any other suitable ligament. In some embodiments, the data representing the patient's soft tissue features include data on a patient's annulus fibrosus, fibrocartilage, nerves, tendons, muscles and the like. The system can generate predicted intraoperative mobility data (e.g., single level intraoperative mobility, multi-level intraoperative mobility, etc.) based on the spine configuration and partial or complete severing of the soft tissue.

The client computing device 102 is operably connected via a communication network 104 to a server 106, thus allowing for data transfer between the client computing device 102 and the server 106. The communication network 104 may be a wired and/or a wireless network. The communication network 104, if wireless, may be implemented using communication techniques such as Visible Light Communication (VLC), Worldwide Interoperability for Microwave Access (WiMAX), Long term evolution (LTE), Wireless local area network (WLAN), Infrared (IR) communication, Public Switched Telephone Network (PSTN), Radio waves, and/or other communication techniques known in the art.

The server 106, which may also be referred to as a “treatment assistance network” or “prescriptive analytics network,” can include one or more computing devices and/or systems. As discussed further herein, the server 106 can include one or more processors, and memory storing instructions executable by the one or more processors to perform the methods described herein. In some embodiments, the server 106 is implemented as a distributed “cloud” computing system or facility across any suitable combination of hardware and/or virtual computing resources.

The client computing device 102 and server 106 can individually or collectively perform the various methods described herein for providing patient-specific medical care. For example, some or all of the steps of the methods described herein can be performed by the client computing device 102 alone, the server 106 alone, or a combination of the client computing device 102 and the server 106. Thus, although certain operations are described herein with respect to the server 106, it shall be appreciated that these operations can also be performed by the client computing device 102, and vice-versa.

The server 106 includes at least one database 110 configured to store reference data useful for the treatment planning methods described herein. The reference data can include historical and/or clinical data from the same and/or other patients, data collected from prior surgeries and/or other treatments of patients by the same or other healthcare providers, data relating to medical device designs, data collected from study groups or research groups, data from practice databases, data from academic institutions, data from implant manufacturers or other medical device manufacturers, data from imaging studies, data from simulations, clinical trials, demographic data, treatment data, outcome data, mortality rates, or the like.

In some embodiments, the database 110 includes a plurality of reference patient data sets, each patient reference data set associated with a corresponding reference patient. For example, the reference patient can be a patient that previously received treatment or is currently receiving treatment. Each reference patient data set can include data representative of the corresponding reference patient's condition, anatomy, pathology, medical history, disease progression, preferences, and/or any other information or parameters relevant to the reference patient, such as any of the data described herein with respect to the patient data set 108. In some embodiments, the reference patient data set includes pre-operative data, intraoperative data, and/or post-operative data. For example, a reference patient data set can include data representing one or more of patient ID, age, gender, BMI, lumbar lordosis, Cobb angle(s), pelvic incidence, disc height, segment flexibility, bone quality, rotational displacement, soft tissue features, and/or treatment level of the spine. As another example, a reference patient data set can include treatment data regarding at least one treatment procedure performed on the reference patient, such as descriptions of surgical procedures or interventions (e.g., surgical approaches, bony resections, surgical maneuvers, corrective maneuvers, placement of implants or other devices). In some embodiments, the treatment data includes medical device design data for at least one medical device used to treat the reference patient, such as physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties). In yet another example, a reference patient data set can include outcome data representing an outcome of the treatment of the reference patient, such as corrected anatomical metrics, presence of fusion, HRQL, activity level, return to work, complications, recovery times, efficacy, mortality, and/or follow-up surgeries.

In some embodiments, the server 106 receives at least some of the reference patient data sets from a plurality of healthcare provider computing systems 112 (e.g., also referred to as the “healthcare provider computing systems 112a-112c”). The server 106 can be connected to the healthcare provider computing systems 112 via one or more communication networks (not shown). Each of the healthcare provider computing systems 112a-112c can be associated with a corresponding healthcare provider (e.g., physician, surgeon, medical clinic, hospital, healthcare network, etc.). Each of the healthcare provider computing systems 112a-112c can include at least one reference patient data set (e.g., also referred to as the “reference patient data sets 114a-114c”) associated with reference patients treated by the corresponding healthcare provider. The reference patient data sets 114 can include, for example, electronic medical records, electronic health records, biomedical data sets, etc. The reference patient data sets 114 can be received by the server 106 from the healthcare provider computing systems 112 and can be reformatted into different formats for storage in the database 110. Optionally, the reference patient data sets 114 can be processed (e.g., cleaned) to ensure that the represented patient parameters are likely to be useful in the treatment planning methods described herein.

As described in further detail herein, the server 106 can be configured with one or more algorithms that generate patient-specific treatment plan data (e.g., treatment procedures, medical devices) based on the reference data. In some embodiments, the patient-specific data is generated based on correlations between the patient data set 108 and the reference data. Additionally, or alternatively, the server 106 can identify one or more adjustments to treatment plans. For example, the server 106 can identify one or more additional steps for a medical procedure that affect intraoperative mobility of the patient's anatomical features being treated. In some embodiments, the additional steps can provide secondary corrective treatment to the patient's anatomy that improves outcomes for the patient. Additionally, or alternatively, the server 106 can predict outcomes from the treatment plans and/or the one or more identified additional steps, including recovery times, efficacy based on clinical end points, likelihood of success, predicted mortality, predicted related follow-up surgeries, or the like. In some embodiments, the server 106 can continuously or periodically analyze patient data (including patient data obtained during the patient stay) to determine near real-time or real-time risk scores, mortality prediction, etc.

In some embodiments, the server 106 includes one or more modules for performing one or more steps of the patient-specific treatment planning methods described herein. For example, in the depicted embodiment, the server 106 includes a data analysis module 116 and a treatment planning module 118. In alternative embodiments, one or more of these modules may be combined with each other, or may be omitted. Thus, although certain operations are described herein with respect to a particular module or modules, this is not intended to be limiting, and such operations can be performed by a different module or modules in alternative embodiments.

The data analysis module 116 can be configured with one or more algorithms to generate a virtual model of the patient's anatomical features from the patient data. In some embodiments, for example, the data analysis module 116 can compile image data to generate a three-dimensional (3D) virtual model of the patient's bone structure, then over lay the 3D virtual model with data on the patient's soft tissue features. The 3D virtual model can allow a surgeon or other medical provider to visualize the patient's current anatomy, as well as visualize surgical steps identified by the server 106.

The data analysis module 116 can be configured with one or more algorithms for identifying a subset of reference data from the database 110 that is likely to be useful in developing a patient-specific treatment plan. For example, the data analysis module 116 can compare patient-specific data (e.g., the patient data set 108 received from the client computing device 102) to the reference data from the database 110 (e.g., the reference patient data sets) to identify similar data (e.g., one or more similar patient data sets in the reference patient data sets). The comparison can be based on one or more parameters, such as age, gender, BMI, lumbar lordosis, pelvic incidence, and/or treatment levels. The parameter(s) can be used to calculate a similarity score for each reference patient. The similarity score can represent a statistical correlation between the patient data set 108 and the reference patient data set. Accordingly, similar patients can be identified based on whether the similarity score is above, below, or at a specified threshold value. For example, as described in greater detail below, the comparison can be performed by assigning values to each parameter and determining the aggregate difference between the subject patient and each reference patient. Reference patients whose aggregate difference are below a threshold can be considered to be similar patients.

The data analysis module 116 can further be configured with one or more algorithms to select a subset of the reference patient data sets, e.g., based on similarity to the patient data set 108 and/or treatment outcome of the corresponding reference patient. For example, the data analysis module 116 can identify one or more similar patient data sets in the reference patient data sets, and then select a subset of the similar patient data sets based on whether the similar patient data set includes data indicative of a favorable or desired treatment outcome. The outcome data can include data representing one or more outcome parameters, such as corrected anatomical metrics, presence of fusion, HRQL, activity level, complications, recovery times, efficacy, mortality, or follow-up surgeries. As described in further detail below, in some embodiments, the data analysis module 116 calculates an outcome score by assigning values to each outcome parameter. A patient can be considered to have a favorable outcome if the outcome score is above, below, or at a specified threshold value.

In some embodiments, the data analysis module 116 selects a subset of the reference patient data sets based at least in part on user input (e.g., from a clinician, surgeon, physician, healthcare provider). For example, the user input can be used in identifying similar patient data sets. In some embodiments, weighting of similarity and/or outcome parameters can be selected by a healthcare provider or physician to adjust the similarity and/or outcome score based on clinician input. In further embodiments, the healthcare provider or physician can select the set of similarity and/or outcome parameters (or define new similarity and/or outcome parameters) used to generate the similarity and/or outcome score, respectively.

In some embodiments, the data analysis module 116 includes one or more algorithms used to select a set or subset of the reference patient data sets based on criteria other than patient parameters. For example, the one or more algorithms can be used to select the subset based on healthcare provider parameters (e.g., based on healthcare provider ranking/scores such as hospital/physician expertise, number of procedures performed, hospital ranking, etc.) and/or healthcare resource parameters (e.g., diagnostic equipment, facilities, surgical equipment such as surgical robots), or other non-patient related information that can be used to predict outcomes and risk profiles for procedures for the present healthcare provider. For example, reference patient data sets with images captured from similar diagnostic equipment can be aggregated to reduce or limit irregularities due to variation between diagnostic equipment. Additionally, patient-specific treatment plans can be developed for a particular healthcare provider using data from similar healthcare providers (e.g., healthcare providers with traditionally similar outcomes, physician expertise, surgical teams, etc.). In some embodiments, reference healthcare provider data sets, hospital data sets, physician data sets, surgical team data sets, post-treatment data set, and other data sets can be utilized. By way of example, a patient-specific treatment plan to perform a battlefield surgery can be based on reference patient data from similar battlefield surgeries and/or datasets associated with battlefield surgeries. In another example, the patient-specific treatment plan can be generated based on available surgical robot systems. The reference patient data sets can be selected based on patients that have been operated on using comparable surgical robot systems under similar conditions (e.g., size and capabilities of surgical teams, hospital resources, etc.).

The treatment planning module 118 can be configured with one or more algorithms to generate at least one treatment plan (e.g., pre-operative plans, surgical plans, post-operative plans, additional steps in a prescribed surgical plan, additional steps for a prescribed surgical plan, ancillary surgical procedures for a primary medical treatment, etc.) based on the output from the data analysis module 116. The surgical plan can be designed to achieve a corrected target anatomical configuration and includes one or more soft tissue surgical steps. The soft tissue surgical steps can facilitate movement of anatomical features to the corrected anatomical configuration. The soft tissue surgical steps can also include severing, dissecting, cutting, and/or removing tissue. For example, ligaments (e.g., supraspinous ligament, interspinous ligaments, spinal ligaments, etc.) can be severed to access and move apart adjacent spinous processes. In some example plans, the soft tissue surgical steps include one or more of severing soft tissue located along the patient's spine, removing at least a portion of an annulus, and/or resecting cartilage along the spine. The treatment planning module 118 can virtually move anatomical elements to identify soft tissue that inhibits or prevents desired movement. Simulations of soft tissue surgical steps can be performed to select recommended soft tissue surgical steps for achieving positionability of the anatomical elements.

In some example plans, the soft tissue surgical steps include one or more decompression procedures. The system can predict a decompression score for each decompression procedure. The nerve decompression score can be based on, for example, a predicted percentage decrease of pain felt by the patient. The system can generate a plurality of decompression plans, determine a decompression score (e.g., post-operative pain score, nerve decompression score, etc.) for each decompression plan, receive selection of one of the decompression plans, and generate a decompression surgical plan based on the selected decompression plan. The user can modify the selected decompression plan based on a corrected configuration of the patient's spine. The decompression plans can include at least one of a laminectomy, a laminotomy, a microdiscectomy, a foraminotomy, and/or an osteophyte procedure.

The amount of movement of anatomical elements attributable to each step can be predicted to facilitate surgical planning and simulations. A simulation can predict joint mobility of the patient's spine or specific joints. A user can select one or more of the identified soft tissue surgical steps based on the simulated joint mobility. The treatment planning module 118 can predict intra-operative joint mobility and/or post-operative joint mobility associated with the selected soft tissue surgical steps. This allows the user to select a surgical plan with soft tissue surgical steps for helping reposition anatomical elements.

In some embodiments, the treatment planning module 118 is configured to develop and/or implement at least one predictive model for generating the patient-specific treatment plan and/or predicting an outcome of the treatment plan. The predictive model(s) can be developed using clinical knowledge, statistics, machine learning, AI, neural networks, or the like. In some embodiments, the output from the data analysis module 116 is analyzed (e.g., using statistics, machine learning, neural networks, AI) to identify correlations between data sets, patient parameters, healthcare provider parameters, healthcare resource parameters, treatment procedures, medical device designs, and/or treatment outcomes. These correlations can be used to develop at least one predictive model that predicts the likelihood that a generated treatment plan and/or will produce a favorable outcome for the particular patient and/or an effect of the one or more additional steps will have on the outcome. The predictive model(s) can be validated, e.g., by inputting data into the model(s) and comparing the output of the model to the expected output.

In some embodiments, the treatment planning module 118 is configured to generate and/or update the treatment plan based on previous treatment data from reference patients. For example, the treatment planning module 118 can receive a selected subset of reference patient data sets and/or similar patient data sets from the data analysis module 116, and determine or identify treatment data from the selected subset. The treatment data can include, for example, treatment procedure data (e.g., surgical procedure or intervention data and/or data on the surgeon's technique data) and/or medical device design data (e.g. implant design data) that is associated with favorable or desired treatment outcomes for the corresponding patient. The treatment planning module 118 can analyze the treatment procedure data, additional identified steps for the procedure, and/or medical device design data to determine an optimal treatment protocol for the patient to be treated. For example, the treatment procedures, additional identified steps for the procedure, and/or medical device designs can be assigned values and aggregated to produce a treatment score. The patient-specific treatment plan can be determined by selecting treatment plan(s) based on the score (e.g., higher or highest score; lower or lowest score; score that is above, below, or at a specified threshold value). The personalized patient-specific treatment plan can be based on, at least in part, the patient-specific technologies or patient-specific selected technology.

Alternatively, or in combination, the treatment planning module 118 can generate the treatment plan and/or identify additional steps based on correlations between data sets. For example, the treatment planning module 118 can correlate treatment procedure data and/or intraoperatively received data from similar patients with favorable outcomes (e.g., as identified by the data analysis module 116). Correlation analysis can include transforming correlation coefficient values to values or scores. The values/scores can be aggregated, filtered, or otherwise analyzed to determine one or more statistical significances. These correlations can be used to determine treatment procedure(s) and/or medical device design(s) that are optimal or likely to produce a favorable outcome for the patient to be treated.

Alternatively, or in combination, the treatment planning module 118 can generate the treatment plan and/or identify additional steps using one or more AI techniques. AI techniques can be used to develop computing systems capable of simulating aspects of human intelligence, e.g., learning, reasoning, planning, problem solving, decision making, etc. AI techniques can include, but are not limited to, case-based reasoning, rule-based systems, artificial neural networks, decision trees, support vector machines, regression analysis, Bayesian networks (e.g., naïve Bayes classifiers), genetic algorithms, cellular automata, fuzzy logic systems, multi-agent systems, swarm intelligence, data mining, machine learning (e.g., supervised learning, unsupervised learning, reinforcement learning), and hybrid systems.

In some embodiments, the treatment planning module 118 generates the treatment plan using one or more trained machine learning models. Various types of machine learning models, algorithms, and techniques are suitable for use with the present technology. In some embodiments, the machine learning model is initially trained on a training data set, which is a set of examples used to fit the parameters (e.g., weights of connections between “neurons” in artificial neural networks) of the model. For example, the training data set can include any of the reference data stored in database 110, such as a plurality of reference patient data sets or a selected subset thereof (e.g., a plurality of similar patient data sets).

In some embodiments, the machine learning model (e.g., a neural network or a naïve Bayes classifier) may be trained on the training data set using a supervised learning method (e.g., gradient descent or stochastic gradient descent). The training dataset can include pairs of generated “input vectors” with the associated corresponding “answer vector” (commonly denoted as the target). The current model is run with the training data set and produces a result, which is then compared with the target, for each input vector in the training data set. Based on the result of the comparison and the specific learning algorithm being used, the parameters of the model are adjusted. The model fitting can include both variable selection and parameter estimation. The fitted model can be used to predict the responses for the observations in a second data set called the validation data set. The validation data set can provide an unbiased evaluation of a model fit on the training data set while tuning the model parameters. Validation data sets can be used for regularization by early stopping, e.g., by stopping training when the error on the validation data set increases, as this may be a sign of overfitting to the training data set. In some embodiments, the error of the validation data set error can fluctuate during training, such that ad-hoc rules may be used to decide when overfitting has truly begun. Finally, a test data set can be used to provide an unbiased evaluation of a final model fit on the training data set.

To generate a treatment plan, the patient data set 108 can be input into the trained machine learning model(s). Additional data, such as the selected subset of reference patient data sets and/or similar patient data sets, treatment data from the selected subset, and/or intraoperatively obtained patient data sets can also be input into the trained machine learning model(s). The trained machine learning model(s) can then calculate whether various candidate treatment procedures, additional steps for a planned treatment procedure, and/or medical device designs are likely to produce a favorable outcome for the patient. Based on these calculations, the trained machine learning model(s) can select at least one treatment plan for the patient. In embodiments where multiple trained machine learning models are used, the models can be run sequentially or concurrently to compare outcomes and can be periodically updated using training data sets and/or updated patient data sets. The treatment planning module 118 can use one or more of the machine learning models based at least partially on a predicted accuracy score for the model.

A patient-specific treatment plan generated by the treatment planning module 118 can include at least one patient-specific treatment procedure (e.g., a surgical procedure or intervention) and/or at least one patient-specific medical device (e.g., an implant or implant delivery instrument); an update to a patient-specific treatment plan generated by the treatment planning module 118 can include at least one additional step for a patient-specific treatment procedure and/or at least one modification to a patient-specific medical device. A patient-specific treatment plan can include an entire surgical procedure or portions thereof. Similarly, an update to the patient-specific treatment plan can include a single additional step to the surgical procedure, portions thereof, multiple options for additional steps, and/or an entire surgical procedure. Additionally, one or more patient-specific medical devices can be specifically selected or designed for the corresponding surgical procedure, thus allowing for the various components of the patient-specific technology to be used in combination to treat the patient.

In some embodiments, the patient-specific treatment procedure includes an orthopedic surgery procedure, such as spinal surgery, hip surgery, knee surgery, jaw surgery, hand surgery, shoulder surgery, elbow surgery, total joint reconstruction (arthroplasty), skull reconstruction, foot surgery, or ankle surgery. Spinal surgery can include spinal fusion surgery, such as lumbar interbody fusion (LIF), posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). In some embodiments, the patient-specific treatment procedure and/or updates thereof include descriptions of and/or instructions for performing one or more aspects of a patient-specific surgical procedure and/or additional steps to the patient-specific surgical procedure. For example, the patient-specific surgical procedure can include one or more of a surgical approach, a corrective maneuver, a bony resection, or implant placement. In another example, an update to the patient-specific surgical procedure can include instructions to remove additional tissue at or around the treatment site, remove bone anomalies (e.g., bridging osteophytes or autofused segments) at ancillary sites adjacent the primary treatment site, or the like.

In some embodiments, the patient-specific medical device design includes a design for an orthopedic implant and/or a design for an instrument for delivering an orthopedic implant. Examples of such implants include, but are not limited to, screws (e.g., bone screws, spinal screws, pedicle screws, facet screws), interbody implant devices (e.g., intervertebral implants), intervertebral body fusion (“IBF”) devices, interspinous spacers, cages, plates, endplates, rods, disks, fusion devices, spacers, rods, expandable devices, stents, brackets, ties, scaffolds, fixation device, anchors, nuts, bolts, rivets, connectors, tethers, fasteners, joint replacements, hip implants, or the like. Examples of instruments include, but are not limited to, screw guides, cannulas, ports, catheters, insertion tools, decompression instruments, or the like.

A patient-specific medical device design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of a corresponding medical device. For example, a design for an orthopedic implant can include implant shape, size, material, and/or effective stiffness (e.g., lattice density, number of struts, location of struts, etc.). In some embodiments, the generated patient-specific medical device design is a design for an entire device. Alternatively, the generated design can be for one or more components of a device, rather than the entire device.

In some embodiments, the design is for one or more patient-specific device components that can be used with standard, off-the-shelf components. For example, in a spinal surgery, an IBF device can include both standard components and patient-specific customized components. In some embodiments, the generated design is for a patient-specific medical device that can be used with a standard, off-the-shelf delivery instrument. For example, the implants (e.g., endplates, expansion devices, screws) can be designed and manufactured for the patient, (e.g., to match the patient's anatomy and/or to account for the patient-specific medical procedure) while the instruments for delivering the implants can be standard instruments. This approach allows the components that are implanted to be designed and manufactured based on the patient's anatomy and/or surgeon's preferences to enhance treatment. The patient-specific devices described herein are expected to improve delivery into the patient's body, placement at the treatment site, and/or interaction with the patient's anatomy.

In embodiments where the patient-specific treatment plan includes a surgical procedure to implant a medical device, the treatment planning module 118 can also store various types of implant surgery information, such as implant parameters (e.g., types, dimensions), availability of implants, aspects of a pre-operative plan (e.g., initial implant configuration, detection and measurement of the patient's anatomy, etc.), FDA requirements for implants (e.g., specific implant parameters and/or characteristics for compliance with FDA regulations), or the like. In some embodiments, the treatment planning module 118 can convert the implant surgery information into formats useable for machine-learning based models and algorithms. For example, the implant surgery information can be tagged with particular identifiers for formulas or can be converted into numerical representations suitable for supplying to the trained machine learning model(s). The treatment planning module 118 can also store information regarding the patient's anatomy, such as two- or three-dimensional images or models of the anatomy, and/or information regarding the biology, geometry, and/or mechanical properties of the anatomy. The anatomy information can be used to inform implant design and/or placement.

The treatment plan(s) generated by the treatment planning module 118 can be transmitted via the communication network 104 to the client computing device 102 for output to a user (e.g., clinician, surgeon, healthcare provider, patient). In some embodiments, the client computing device 102 includes or is operably coupled to a display 122 for outputting the treatment plan(s) and/or the virtual model of the patient's anatomical features generated by the data analysis module 116. The display 122 can include a graphical user interface (GUI) for visually depicting various aspects of the treatment plan(s) and/or the virtual model. For example, the display 122 can show various aspects of a surgical procedure to be performed on the patient, such as the surgical approach, treatment levels, corrective maneuvers, tissue resection, and/or implant placement. To facilitate visualization, the virtual model of patient's anatomical features can be integrated with the surgical procedure for display. As another example, the display 122 can show a device design 135 for a medical device to be implanted in the patient, such as a two- or three-dimensional model of the device design 135. The display 122 can also show the virtual model, in two- or three-dimensional images, of the patient's anatomy where the surgical procedure is to be performed and/or where the device is to be implanted. The client computing device 102 can further include one or more user input devices (not shown) allowing the user to modify, select, approve, and/or reject the displayed treatment plan(s).

In some embodiments, the medical device design(s) generated by the treatment planning module 118 can be transmitted from the client computing device 102 and/or server 106 to a manufacturing system 124 for manufacturing a corresponding medical device. The manufacturing system 124 can be located on site or off site. On-site manufacturing can reduce the number of sessions with a patient and/or the time to be able to perform the surgery whereas off-site manufacturing can be useful to make the complex devices. Off-site manufacturing facilities can have specialized manufacturing equipment. In some embodiments, more complicated device components can be manufactured off site, while simpler device components can be manufactured on site.

Various types of manufacturing systems are suitable for use in accordance with the embodiments herein. For example, the manufacturing system 124 can be configured for additive manufacturing, such as 3D printing, stereolithography (SLA), digital light processing (DLP), fused deposition modeling (FDM), selective laser sintering (SLS), selective laser melting (SLM), selective heat sintering (SHM), electronic beam melting (EBM), laminated object manufacturing (LOM), powder bed printing (PP), thermoplastic printing, direct material deposition (DMD), inkjet photo resin printing, or like technologies, or combination thereof. Alternatively or in combination, the manufacturing system 124 can be configured for subtractive (traditional) manufacturing, such as CNC machining, electrical discharge machining (EDM), grinding, laser cutting, water jet machining, manual machining (e.g., milling, lathe/turning), or like technologies, or combinations thereof. The manufacturing system 124 can manufacture one or more patient-specific medical devices based on fabrication instructions or data (e.g., CAD data, 3D data, digital blueprints, stereolithography data, or other data suitable for the various manufacturing technologies described herein). Different components of the system 100 can generate at least a portion of the manufacturing data used by the manufacturing system 124. The manufacturing data can include, without limitation, fabrication instructions (e.g., programs executable by additive manufacturing equipment, subtractive manufacturing equipment, etc.), 3D data, CAD data (e.g., CAD files), CAM data (e.g., CAM files), path data (e.g., print head paths, tool paths, etc.), material data, tolerance data, surface finish data (e.g., surface roughness data), regulatory data (e.g., FDA requirements, reimbursement data, etc.), or the like. The manufacturing system 124 can analyze the manufacturability of the implant design based on the received manufacturing data. The implant design can be finalized by altering geometries, surfaces, etc. and then generating manufacturing instructions. In some embodiments, the server 206 generates at least a portion of the manufacturing data, which is transmitted to the manufacturing system 124.

The manufacturing system 124 can generate CAM data, print data (e.g., powder bed print data, thermoplastic print data, photo resin data, etc.), or the like and can include additive manufacturing equipment, subtractive manufacturing equipment, thermal processing equipment, or the like. The additive manufacturing equipment can be 3D printers, stereolithography devices, digital light processing devices, fused deposition modeling devices, selective laser sintering devices, selective laser melting devices, electronic beam melting devices, laminated object manufacturing devices, powder bed printers, thermoplastic printers, direct material deposition devices, or inkjet photo resin printers, or like technologies. The subtractive manufacturing equipment can be CNC machines, electrical discharge machines, grinders, laser cutters, water jet machines, manual machines (e.g., milling machines, lathes, etc.), or like technologies. Both additive and subtractive techniques can be used to produce implants with complex geometries, surface finishes, material properties, etc. The generated fabrication instructions can be configured to cause the manufacturing system 124 to manufacture the patient-specific orthopedic implant that matches or is therapeutically the same as the patient-specific design. In some embodiments, the patient-specific medical device can include features, materials, and designs shared across designs to simplify manufacturing. For example, deployable patient-specific medical devices for different patients can have similar internal deployment mechanisms but have different deployed configurations. In some embodiments, the components of the patient-specific medical devices are selected from a set of available pre-fabricated components and the selected pre-fabricated components can be modified based on the fabrication instructions or data.

The treatment plans described herein can be performed by a surgeon, a surgical robot, or a combination thereof, thus allowing for treatment flexibility. In some embodiments, the surgical procedure can be performed entirely by a surgeon, entirely by a surgical robot, or a combination thereof. For example, one step of a surgical procedure can be manually performed by a surgeon and another step of the procedure can be performed by a surgical robot. In some embodiments the treatment planning module 118 generates control instructions configured to cause a surgical robot (e.g., robotic surgery systems, navigation systems, etc.) to partially or fully perform a surgical procedure. The control instructions can be transmitted to the robotic apparatus by the client computing device 102 and/or the server 106.

Following the treatment of the patient in accordance with the treatment plan, treatment progress can be monitored over one or more time periods to update the data analysis module 116 and/or treatment planning module 118. Post-treatment data can be added to the reference data stored in the database 110. The post-treatment data can be used to train machine learning models for developing patient-specific treatment plans, patient-specific medical devices, or combinations thereof.

It shall be appreciated that the components of the system 100 can be configured in many different ways. For example, in alternative embodiments, the database 110, the data analysis module 116 and/or the treatment planning module 118 can be components of the client computing device 102, rather than the server 106. As another example, the database 110 the data analysis module 116, and/or the treatment planning module 118 can be located across a plurality of different servers, computing systems, or other types of cloud-computing resources, rather than at a single server 106 or client computing device 102.

Additionally, in some embodiments, the system 100 can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, programmable consumer electronics, network PCs, minicomputers, mainframe computers, distributed computing environments that include any of the above systems or devices, or the like.

FIG. 2 illustrates a computing device 200 suitable for use in connection with the system 100 of FIG. 1 according to some embodiments of the present technology. The computing device 200 can be incorporated in various components of the system 100 of FIG. 1, such as the client computing device 202 or the server 206. The computing device 200 includes one or more processors 210 (e.g., CPU(s), GPU(s), HPU(s), etc.). The processor(s) 210 can be a single processing unit or multiple processing units in a device or distributed across multiple devices. The processor(s) 210 can be coupled to other hardware devices, for example, with the use of a bus, such as a PCI bus or SCSI bus. The processor(s) 210 can be configured to execute one more computer-readable program instructions, such as program instructions to carry out of any of the methods described herein.

The computing device 200 can include one or more input devices 220 that provide input to the processor(s) 210, e.g., to notify it of actions from a user of the computing device 200. The actions can be mediated by a hardware controller that interprets the signals received from the input device and communicates the information to the processor(s) 210 using a communication protocol. Input device(s) 220 can include, for example, a mouse, a keyboard, a touchscreen, an infrared sensor, a touchpad, a wearable input device, a camera- or image-based input device, a microphone, or other user input devices.

The computing device 200 can include a display 230 used to display various types of output, such as text, virtual models, virtual procedures, surgical plans, implants, graphics, and/or images (e.g., images with voxels indicating radiodensity units or Hounsfield units representing the density of the tissue at a location, such as local densities of bone or soft tissue). In some embodiments, the display 230 provides graphical and textual visual feedback to a user. The processor(s) 210 can communicate with the display 230 via a hardware controller for devices. In some embodiments, the display 230 includes the input device(s) 220 as part of the display 230, such as when the input device(s) 220 includes a touchscreen or is equipped with an eye direction monitoring system. In alternative embodiments, the display 230 is separate from the input device(s) 220. Examples of display devices include an LCD display screen, an LED display screen, a projected, holographic, or augmented reality display (e.g., a heads-up display device or a head-mounted device), and so on.

Optionally, other I/O devices 240 can also be coupled to the processor(s) 210, such as a network card, video card, audio card, USB, firewire or other external device, camera, printer, speakers, CD-ROM drive, DVD drive, disk drive, or Blu-Ray device. Other I/O devices 240 can also include input ports for information from directly connected medical equipment such as imaging apparatuses, including MRI machines, X-Ray machines, CT machines, etc. Other I/O devices 240 can further include input ports for receiving data from these types of machine from other sources, such as across a network or from previously captured data, for example, stored in a database.

In some embodiments, the computing device 200 also includes a communication device (not shown) capable of communicating wirelessly or wire-based with a network node. The communication device can communicate with another device or a server through a network using, for example, TCP/IP protocols. The computing device 200 can utilize the communication device to distribute operations across multiple network devices, including imaging equipment, manufacturing equipment, etc.

The computing device 200 can include memory 250, which can be in a single device or distributed across multiple devices. Memory 250 includes one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory. In some embodiments, the memory 250 is a non-transitory computer-readable storage medium that stores, for example, programs, software, data, or the like. In some embodiments, memory 250 can include program memory 260 that stores programs and software, such as an operating system 262, one or more surgical procedure modules 264, and other application programs 266. The surgical procedure module(s) 264 can include one or more modules configured to perform the various methods described herein (e.g., the data analysis module 216 and/or treatment planning module 218 described with respect to FIG. 1). Memory 250 can also include data memory 270 that can include, e.g., patient data, reference data, configuration data, settings, user options or preferences, etc., which can be provided to the program memory 260 or any other element of the computing device 200.

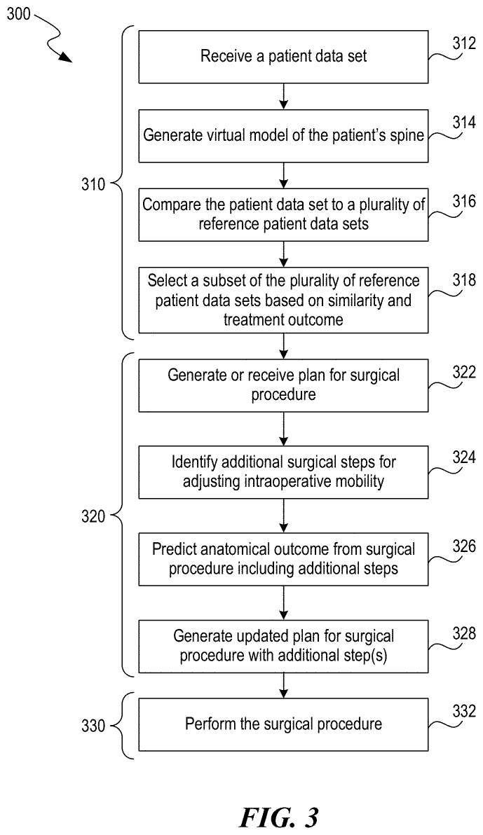

FIG. 3 is a flow diagram illustrating a method 300 for providing patient-specific medical care, according to some embodiments of the present technology. In the illustrated embodiment, The method 300 includes a data phase 310, a modeling and prediction phase 320, and an execution phase 330. The data phase 310 can include collecting data of a patient to be treated (e.g., pathology data, patient data, image data, soft tissue data, and the like), and comparing the patient data to reference data (e.g., prior patient data including as pathology, patient data, image data, soft tissue data, surgical data, and/or outcome data). For example, a patient data set can be received (block 312). The patient data set can then be used to generate a virtual model of the patient's anatomical features (block 314), such as a virtual model of the patient's spine and surrounding soft tissue. The patient data set and/or virtual model can be compared to a plurality of reference patient data sets (block 316), e.g., in order to identify one or more similar patient data sets in the plurality of reference patient data sets. Each of the plurality of reference patient data sets can include data representing one or more of age, gender, BMI, lumbar lordosis, Cobb angle(s), pelvic incidence, disc height, segment flexibility, bone quality, rotational displacement, treatment level of the spine, ligament development, ligament flexibility or extension, or muscle development.

A subset of the plurality of reference patient data sets can be selected (block 318), e.g., based on similarity to the patient data set and/or treatment outcomes of the corresponding reference patients. For example, a similarity score can be generated for each reference patient data set, based on the comparison of the patient data set and the reference patient data set. The similarity score can represent a statistical correlation between the patient data and the reference patient data set. One or more similar patient data sets can be identified based, at least partly, on the similarity score.

In some embodiments, each patient data set of the selected subset includes and/or is associated with data indicative of a favorable treatment outcome (e.g., a favorable treatment outcome based on a single target outcome, aggregate outcome score, outcome thresholding). The data can include, for example, data representing one or more of corrected anatomical metrics (e.g., correction provided, post-operative mobility, etc.), presence of fusion, health related quality of life, activity level, or complications. In some embodiments, the data is or includes an outcome score, which can be calculated based on a single target outcome, an aggregate outcome, and/or an outcome threshold.

Optionally, the data phase 310 can include identifying or determining, for at least one patient data set of the selected subset (e.g., for at least one similar patient data set), surgical procedure data and/or medical device design data associated with the favorable treatment outcome. The surgical procedure data can include data representing one or more of a surgical approach, a corrective maneuver, a bony resection, medical device placement, or a medical device design. The at least one medical device design can include data representing one or more of physical properties, mechanical properties, or biological properties of a corresponding medical device and/or patient. In some embodiments, the at least one patient-specific medical device design includes a design for an implant or an implant delivery instrument that is customized to the patient's anatomical features and/or the plan for the surgical procedure.

In the modeling and prediction phase 320, a plan for a surgical procedure and/or a design for a medical device is generated or received (block 322). For example, in some embodiments, generating the plan for the surgical procedure can include developing at least one predictive model based on the patient data set and/or selected subset of reference patient data sets (e.g., using statistics, machine learning, neural networks, AI, or the like). The predictive model can be configured to generate the plan for the surgical procedure that accounts for patient-specific features, such as unique features in the patient's anatomy.

In some embodiments, the predictive model includes one or more trained machine learning models that generate, at least partly, the surgical procedure and/or medical device design. For example, the trained machine learning model(s) can determine a plurality of candidate surgical procedures and/or medical device designs for treating the patient. Each surgical procedure can be associated with a corresponding medical device design. In some embodiments, the surgical procedures and/or medical device designs are determined based on surgical procedure data and/or medical device design data associated with favorable outcomes, as previously described with respect to the data phase 310. For each surgical procedure and/or corresponding medical device design, the trained machine learning model(s) can calculate a probability of achieving a target outcome (e.g., favorable or desired outcome) for the patient. The trained machine learning model(s) can then select at least one surgical procedure and/or corresponding medical device design based, at least partly, on the calculated probabilities.

In some embodiments of block 322, the surgical procedure is received from a surgeon, physician, or other medical provider. In some such embodiments, the plan for the surgical procedure corresponds to a generalized plan for the surgical procedure (e.g., the common steps involved in a procedure) and/or the surgeon's preferred technique for a surgical procedure.

Once the plan for the surgical procedure is generated and/or received, the modeling and prediction phase 320 can include identifying one or more additional steps for the surgical procedure (block 324). The additional steps can alter the intraoperative mobility of the patient's anatomical structure, surgical tools, and/or a design for an implant device. For example, the additional steps can include a dissection of a spinal ligament that allows a portion of the patient's spine being operated on to increase the mobility of vertebral bodies in the portion of the patient's spine. The additional steps can also target ancillary anatomical features to improve post-operative results and take advantage of the access available during the surgical procedure. For example, the additional steps can include an ancillary removal of bridging osteophytes and/or autofused segments adjacent a portion of the patient's spine being operated on. While the removal may be non-critical to the primary surgical procedure, the removal can improve a post-operative outcome mobility of the patient's spine.

After identifying one or more additional steps for the surgical procedure, the modeling and prediction phase 320 can include predicting an anatomical outcome (block 326) from a surgical procedure that incorporates one, a portion of, and/or all of the additional steps identified at block 324. In some embodiments, predicting an outcome of for the surgical procedure can include developing at least one predictive model based on the patient data set and/or selected subset of reference patient data sets (e.g., using statistics, machine learning, neural networks, AI, or the like). The predictive model can be generated by any of the methods discussed above. Once generated, the predictive model can be applied to the patient data and a plan for the surgical procedure including one, a portion of, and/or all of the additional steps. For example, in some embodiments, the predictive model can be applied to the patient data and each combination of the additional steps identified in block 324 integrated into the plan for the surgical procedure. In some embodiments, the predicted outcome can include data representing one or more outcome parameters, such as corrected anatomical metrics, presence of fusion, HRQL, activity level, complications, recovery times, efficacy, mortality, or follow-up surgeries. In some embodiments, the predicted outcome can include predicting outcome score by assigning values to each outcome parameter, allowing a patient and/or a medical provider to quickly assess the predicted outcomes.

Optionally, the modeling and prediction phase 320 can include a step for feedback from a medical provider, such as a surgeon that will be executing the medical procedure. The feedback can include an indication of preferred additional steps and/or tweaks to the additional steps. The feedback can also include a selection of the additional steps to be integrated into the plan for the surgical procedure. After receiving feedback, in some embodiments, the modeling and prediction phase 320 can include identifying one or more further additional steps and predicting an anatomical outcome from a surgical procedure that incorporates the further additional steps.

The modeling and prediction phase 320 then includes generating an updated plan for the surgical procedure (block 328) with one or more (or, in some embodiments, none) of the additional steps for use in the execution phase 330. The execution phase 330 includes performing the surgical procedure (block 334) according to the updated plan. The surgical procedure can be performed manually, by a surgical robot, or a combination thereof. In embodiments where the surgical procedure is performed by a surgical robot, generating the updated plan at block 328 can include generating control instructions configured to cause the surgical robot to perform, at least partly, the patient-specific surgical procedure at block 332. In some embodiments, generating the updated plan at block 328 includes generating a plan for one or more implants based on the updated plan. For example, if the updated plan includes removing one or more irregularities in a vertebral body, the updated plan can also include manufacturing the implant to account for the removals (e.g., with an engaging surface customized to the patient-specific topology after removing the irregularities).

The method 300 can be implemented and performed in various ways. In some embodiments, one or more steps of the method 300 (e.g., the data phase 310 and/or the modeling and prediction phase 320) can be implemented as computer-readable instructions stored in memory and executable by one or more processors of any of the computing devices and systems described herein (e.g., the system 100), or a component thereof (e.g., the client computing device 102 and/or the server 106). Alternatively, one or more steps of the method 300 (e.g., the execution phase 330) can be performed by a healthcare provider (e.g., physician, surgeon), a robotic apparatus (e.g., a surgical robot), a manufacturing system (e.g., manufacturing system 124), or a combination thereof. In some embodiments, one or more steps of the method 300 are omitted (e.g., the execution phase 330).

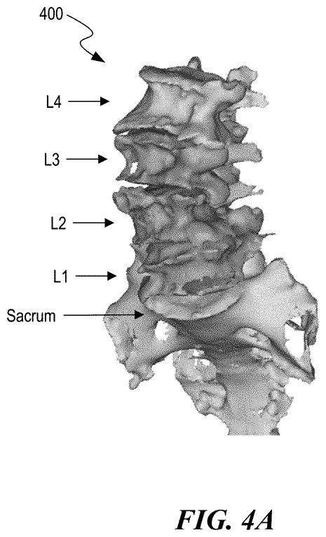

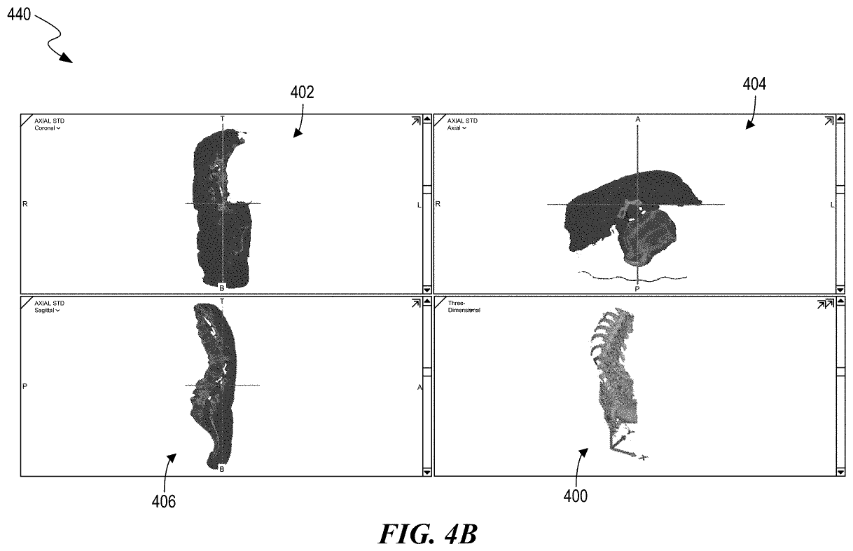

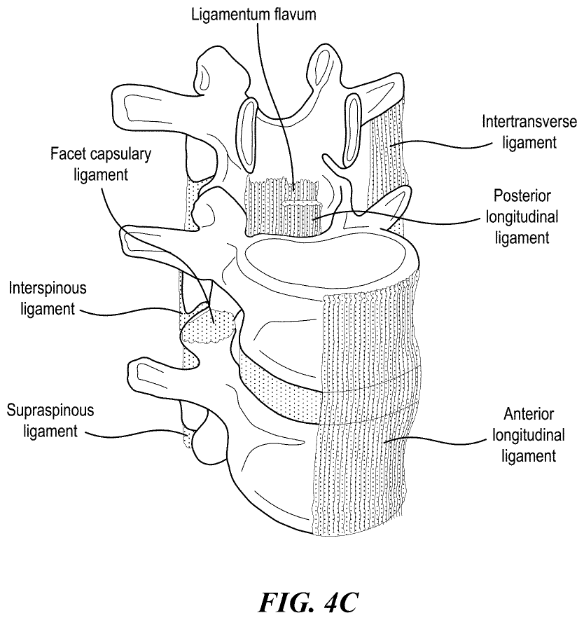

FIGS. 4A-4C illustrate various examples of a virtual model 400 of a patient's native anatomical configuration in accordance with some embodiments of the present technology. In the illustrated embodiment, the virtual model 400 is a 3D visual representation of the patient's native anatomy in various portions of the patient's spine. For example, the virtual model 400 of FIG. 4A includes a 3D visual representation of a portion of the spinal column extending from the sacrum to the L4 vertebral level to the sacrum. Of course, the virtual model 400 can include other regions of the patient's spinal column, including cervical vertebrae, thoracic vertebrae, lumbar vertebrae, and the sacrum. Further, for example as illustrated in FIG. 4C, the virtual model 400 can include additional structures in overlaid with the bony structures illustrated in FIG. 4A, such as cartilage, soft tissue, vascular tissue, nervous tissue, etc.

FIG. 4B illustrates a virtual model display 450 (referred to herein as the “display 450”) showing different views of the virtual model 400. The virtual model display 450 can include a 3D view of the virtual model 400 of FIG. 4A including one or more coronal cross-section(s) 402 of the virtual model 400, one or more axial cross section(s) 404 of the virtual model 400, and/or one or more sagittal cross-section(s) 406 of the virtual model 400. Of course, other views are possible and can be included on the virtual model display 450. Further, as noted above, additional structures (e.g., soft tissue) can be included in the virtual model 400 and included into the display 450. In some embodiments, the virtual model 400 is interactive, allowing a user to manipulate the orientation or view of the virtual model 400 (e.g., rotate), change the depth of the displayed cross-sections, select and isolate specific bony structures, add and/or remove non-bony structures (e.g., cartilage, soft tissue, vascular tissue, nervous tissue, etc.), isolate non-bony structures, or the like.

FIG. 4C illustrates an example of the virtual model 400 that includes soft tissue features 410 overlaid with the bone structure in a portion of the patient's spine. As illustrated, the soft tissue features include the anterior longitudinal ligament, the posterior longitudinal ligament, the intertransverse ligament, the ligament flavum, the facet capsulary ligament, the interspinous ligament, the supraspinous ligament, and the anulus fibrous for a vertebral disc. In some embodiments, the virtual model 400 can allow a user to visualize the one or more additional steps identified by the method 300 of FIG. 3 to visualize the effect of the steps on the mobility of the patient's spine. For example, the virtual model 400 can allow a user to visualize an effect of manipulating any of the soft tissue features in addition to a planned surgical procedure.

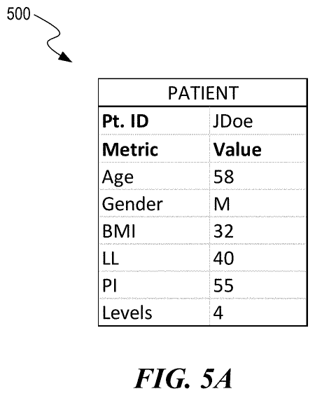

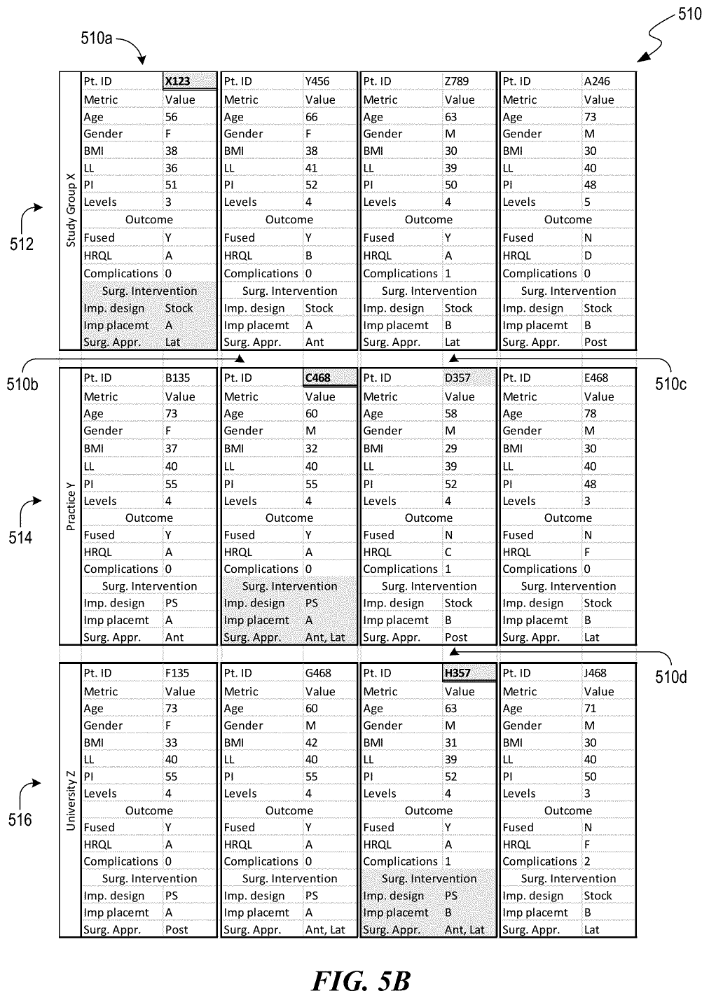

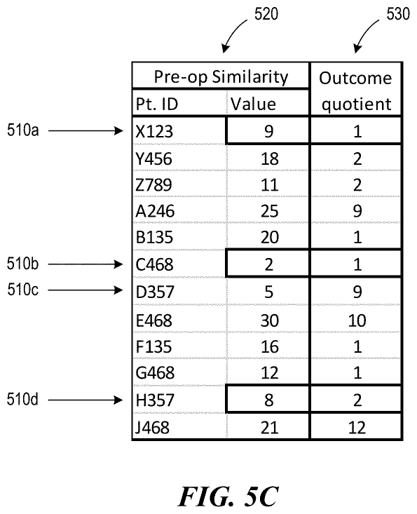

FIGS. 5A-5C illustrate exemplary data sets that may be used and/or generated in connection with the methods described herein (e.g., the data phase 310 described with respect to FIG. 3). FIG. 5A illustrates a patient data set 500 of a patient to be treated. The patient data set 500 can include a patient ID and a plurality of pre-operative patient metrics (e.g., age, gender, BMI, lumbar lordosis (LL), pelvic incidence (PI), and treatment levels of the spine (levels)).

FIG. 5B illustrates a plurality of reference patient data sets 510. In the depicted embodiment, the reference patient data sets 510 include a first subset 512 from a study group (Study Group X), a second subset 514 from a practice database (Practice Y), and a third subset 516 from an academic group (University Z). In alternative embodiments, the reference patient data sets 510 can include data from other sources, as previously described herein. Each reference patient data set can include a patient ID, a plurality of pre-operative patient metrics (e.g., age, gender, BMI, lumbar lordosis (LL), pelvic incidence (PI), and treatment levels of the spine (levels)), treatment outcome data (Outcome) (e.g., presence of fusion (fused), HRQL, complications), and treatment procedure data (Surg. Intervention) (e.g., implant design, implant placement, surgical approach).

FIG. 5C illustrates comparison of the patient data set 500 to the reference patient data sets 510. As previously described, the patient data set 500 can be compared to the reference patient data sets 510 to identify one or more similar patient data sets from the reference patient data sets. In some embodiments, the patient metrics from the reference patient data sets 510 are converted to numeric values and compared the patient metrics from the patient data set 500 to calculate a similarity score 520 (“Pre-op Similarity”) for each reference patient data set. Reference patient data sets having a similarity score below a threshold value can be considered to be similar to the patient data set 500. For example, in the depicted embodiment, reference patient data set 510a has a similarity score of 9, reference patient data set 510b has a similarity score of 2, reference patient data set 510c has a similarity score of 5, and reference patient data set 510d has a similarity score of 8. Because each of these scores are below the threshold value of 20, reference patient data sets 510a-d are identified as being similar patient data sets.

The treatment outcome data of the similar reference patient data sets 510a-d can be analyzed to determine surgical procedures and/or implant designs with the highest probabilities of success. For example, the treatment outcome data for each reference patient data set can be converted to a numerical outcome score 530 (“Outcome Quotient”) representing the likelihood of a favorable outcome. In the depicted embodiment, reference patient data set 510a has an outcome score of 1, reference patient data set 510b has an outcome score of 1, reference patient data set 510c has an outcome score of 9, and reference patient data set 510d has an outcome score of 2. In embodiments where a lower outcome score correlates to a higher likelihood of a favorable outcome, reference patient data sets 510a, 510b, and 510d can be selected. The treatment procedure data from the selected reference patient data sets 510a, 510b, and 510d can then be used to determine at least one surgical procedure (e.g., implant placement, surgical approach) and/or implant design that is likely to produce a favorable outcome for the patient to be treated.

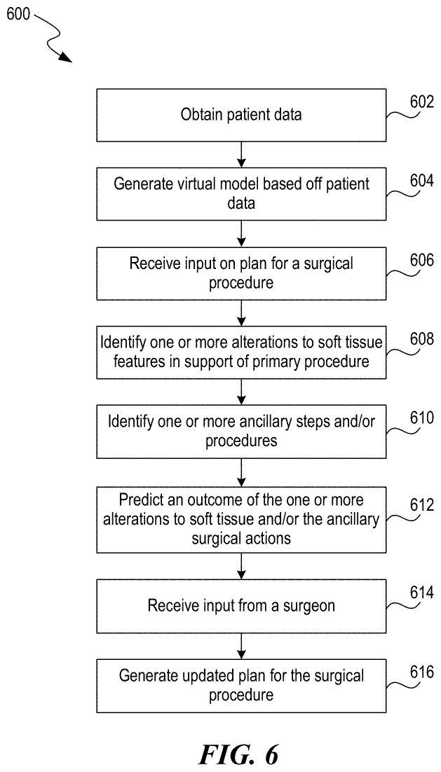

FIGS. 6-8 illustrate various methods 600, 700, 800 for generating and/or updating a patient-specific plan for a surgical procedure in accordance with some embodiments of the present technology. The methods 600, 700, 800 can be implemented and performed in various ways. In some embodiments, one or more steps of the methods 600, 700, 800 can be implemented as computer-readable instructions stored in memory and executable by one or more processors of any of the computing devices and systems described herein (e.g., the system 100), or a component thereof (e.g., the client computing device 102 and/or the server 106). Alternatively, one or more steps of the methods 600, 700, 800 can be performed by a healthcare provider (e.g., physician, surgeon), a robotic apparatus (e.g., a surgical robot), a manufacturing system (e.g., manufacturing system 124), or a combination thereof. In some embodiments, one or more steps of the methods 600, 700, 800 are omitted (e.g., the optional returns illustrated in FIGS. 6-8).

FIG. 6 is a flow diagram illustrating a pre-operative method 600 (“method 600”) for generating a patient-specific plan for a surgical procedure in accordance with some embodiments of the present technology. At block 602, the method 600 includes obtaining patient data. In some embodiments, obtaining the patient data at block 602 includes receiving patient data sets (e.g., image data, pathology data, soft tissue data, etc.) from one or more servers, such as various servers within a medical care network.

At block 604, the method 600 includes generating a virtual model of one or more of the patient's anatomical features based on the patient data. Examples of the resulting vertical model were discussed in more detail above with respect to FIGS. 4A-4C, illustrating that the virtual model provide a healthcare provider with a visualization of the patient's skeletal structures and/or the soft tissue features surrounding them. As discussed above, in some embodiments, the virtual model can be interactive, allowing the healthcare provider to manipulate various anatomical features to visualize the result of the manipulation.

At block 606, the method 600 includes receiving input on a plan for a surgical procedure. The plan for the surgical procedure includes one or more steps configured to address a pathology relevant to the patient's anatomical features. For example, for a patient with scoliosis, the plan for the surgical procedure can include steps to fuse two or more vertebral bodies to at least partially remedy the scoliosis. In various embodiments, the plan for the surgical procedure is received from the healthcare provider and specific to the patient, received from a database and standardized for various pathological conditions relevant to the patient, and/or received from a patient-specific medical care system (e.g., from the server 106 of FIG. 1).

At blocks 608, 610, the method 600 includes identifying one or more additional steps for the plan for the surgical procedure. More specifically, the method 600 includes identifying one or more additional steps that support the received plan for the surgical procedure at block 608; and identifying one or more additional steps and/or procedures that are ancillary to the primary plan for the surgical procedure at block 610. For example, the additional steps identified at block 608 can include soft-tissue related adjustments (e.g., incisions, dissections, manipulations, and the like) that increase intraoperative mobility of the anatomical feature being treated. As a result, the identified additional steps can increase the correction that the surgical procedure provides to the anatomical feature being treated. Similarly, the additional steps identified at block 610 can include soft-tissue related adjustments and/or adjustments to adjacent anatomical structures that improve aspects of an outcome of the surgical procedure. For example, the additional steps identified at block 610 can include incisions, dissections, manipulations, and the like of adjacent anatomical structures that reduce post-operative shifts in the anatomical feature and/or pain associated with correction that the surgical procedure provides to the anatomical feature.

At block 612, the method 600 includes predicting an outcome of various surgical procedures including the additional steps identified at blocks 608, 610. In some embodiments, predicting the outcome includes training a machine learning model, an AI model, a neural network, or the like to predict the anatomical configuration resulting from the surgical procedures that include one or more of the additional steps. For example, as discussed above, reference patient data can be used to train a machine learning model to predict the anatomical effect and/or outcomes associated with one or surgical steps, sometimes isolated to reference patients that have similarities with the subject patient. In some such embodiments, predicting the outcome includes adjusting the virtual model to reflect the predicted anatomical configuration, allowing the healthcare provider and/or patient to visualize the predicted outcome. In some embodiments, predicting the outcome includes defining a desired anatomical configuration and predicting whether surgical procedures that include one or more of the additional steps are likely to achieve the desired anatomical configuration. In some embodiments, predicting the outcome includes generating an outcome score. The outcome score can reflect how closely a predicted anatomical configuration is to the desired anatomical configuration, whether the predicted anatomical configuration is improved by the one or more additional steps, and/or a risk factor associated with the one or more additional steps. The method 600 can include predicting the outcome for surgical procedures including each of the additional steps individually and/or each possible combination of the additional steps.

At block 614, the method 600 includes receiving an input from a surgeon (or other healthcare provider) regarding the additional steps identified at blocks 608, 610. The input can include an identification of risks associated with the additional steps that the method 600 did not recognize, modifications to one or more of the additional steps, a selection of one or more additional steps, an order for the one or more selected additional steps, and/or any other suitable indication.

At block 616, the method 600 includes generating an updated plan for the surgical procedure based on the inputs from the surgeon and the identified and/or selected additional steps. For example, generating the updated plan can include compiling the plan for the surgical procedure based on the received selection of one or more additional steps. In some embodiments, the updated plan is formatted for instruction to the surgeon and/or any other healthcare provider to follow. In some embodiments, one or more steps of the updated plan (up to and including the entire plan), are formatted for execution by a surgical robot and/or any other suitable automated system. For example, in some embodiments, one or more additional soft tissue steps can be formatted for execution by a surgical robot while the surgeon executes another step of the surgical plan. In some embodiments, generating the updated plan for the surgical procedure includes one or more steps to custom-manufacture an implant for the surgical procedure. For example, the implant can be custom-manufactured to support the updated plan for the surgical procedure (e.g., to support a weakened joint during a healing period) an/or to account for the updated plan for the surgical procedure (e.g., customizing one or more surfaces to an expected patient-specific topology after the updated surgical procedure).

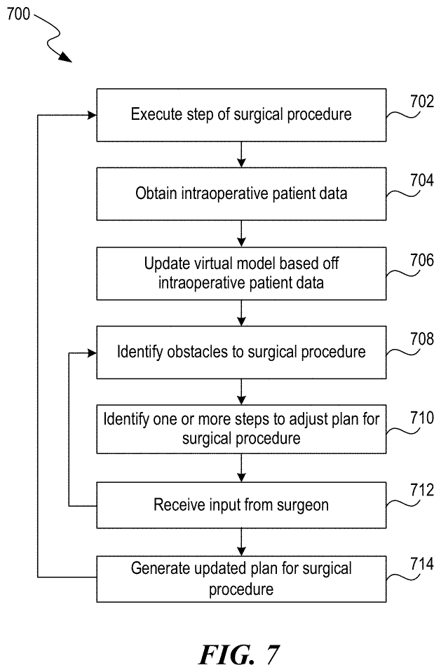

FIG. 7 is a flow diagram illustrating an intraoperative method 700 (“method 700”) for adapting a patient-specific plan for a surgical procedure to patient-specific anatomical structures during the surgical procedure in accordance with some embodiments of the present technology. The method begins at block 702, where the method 700 includes executing one or more steps of a surgical procedure. The step can be executed by a surgeon, other healthcare provider, and/or a surgical robot executing portions of (or all of) a plan for a surgical procedure.

At block 704, the method 700 includes obtaining intraoperative patient data. The interoperative patient data can include intraoperative image data that can provide a more accurate depiction of one or more anatomical features than could be obtained preoperatively. For example, the interoperative patient data can provide an accurate depiction of one or more soft tissue features surrounding a skeletal feature being treated by the surgical procedure. In some embodiments, the interoperative patient data includes a measurement of the anatomical response to the executed step of the surgical procedure. For example, the interoperative patient data can include a measurement of the lordotic and/or coronal correction provided by the executed step of the surgical procedure. The measurement of the anatomical response can increase the efficacy of any intraoperative tweaks to the plan for the surgical procedure.

At block 706, the method 700 includes updating a virtual model of the patient's anatomical configuration based on the intraoperative patient data. In some embodiments, the update to the virtual model includes updating the depicted anatomical features according to the updated patient data. In some embodiments, the update includes updating a predicted anatomical response to manipulation of the patient's anatomical features based on the updated patient data.

At block 708, the method 700 includes identifying one or more obstacles to the plan for the surgical procedure based on the updated virtual model. The identified obstacles can include obstacles to further executing the plan for the surgical procedure (e.g., due to a soft tissue feature blocking an access point), obstacles to achieving the desired anatomical configuration through the surgical procedure (e.g., based on a measured anatomical response below a predicted anatomical response), and the like.

At block 710, the method 700 includes identifying one or more additional steps for the plan for the surgical procedure. In some embodiments, the one or more additional steps identified are responsive to the identified obstacle. For example, if the method 700 identifies an obstacle to achieving the desired anatomical configuration, the method 700 can include identifying additional step(s) that address the obstacle. In a specific, non-limiting example, the method 700 can identify a spinal ligament that is limiting the intraoperative mobility of the patient's spine and thereby preventing the surgical procedure from providing a desired correction to the spinal anatomy. Accordingly, the method 700 can identify one or more points to manipulate (e.g., dissect) the ligament to increase the intraoperative mobility of the patient's spine. In some embodiments, the one or more additional steps identified are responsive to updates in the virtual model. For example, the method 700 can include identifying additional step(s) based on an identification of ancillary anatomical features that can be corrected (e.g., a bridging osteophyte). In another example, the method 700 can include identifying additional step(s) based on an update indicating the patient will respond positively to (or endure) additional corrective actions (e.g., an update to the patient data indicating the patient will respond positively to a more significant anatomical correction or more intensive procedure).

At block 712, the method 700 includes receiving an input from a surgeon (or other healthcare provider) regarding the additional steps identified at block 710 and/or the obstacles identified at block 708. The input can include an identification of risks associated with the additional steps that the method 700 did not recognize, modifications to one or more of the additional steps, one or more additional steps the method 700 did not identify (e.g., addressing the identified obstacles), a selection of one or more additional steps, an input on the operational order for one or more selected additional steps, and/or any other suitable indication.

Optionally, after receiving the input from the surgeon, the method 700 can return to blocks 708, 710 to identify one or more further obstacles and/or additional steps. For example, in embodiments in which a surgeon indicates one or more additional steps addressing an obstacle, the method 700 can return to block 708 to identify any additional obstacles based on the input and/or block 710 to identify additional steps based on the input.

At block 714, the method 700 includes generating an updated plan for the surgical procedure based on the inputs from the surgeon and the additional steps identified and/or selected. As discussed above, the updated plan can be formatted for instruction to the surgeon and/or any other healthcare provider to follow, and/or for execution (partial or entire) by a surgical robot and/or any other suitable automated system. Further, as discussed above, generating the updated plan for the surgical procedure can include one or more steps to custom-manufacture a patient-specific implant (e.g., to account for the updated plan for the surgical procedure).

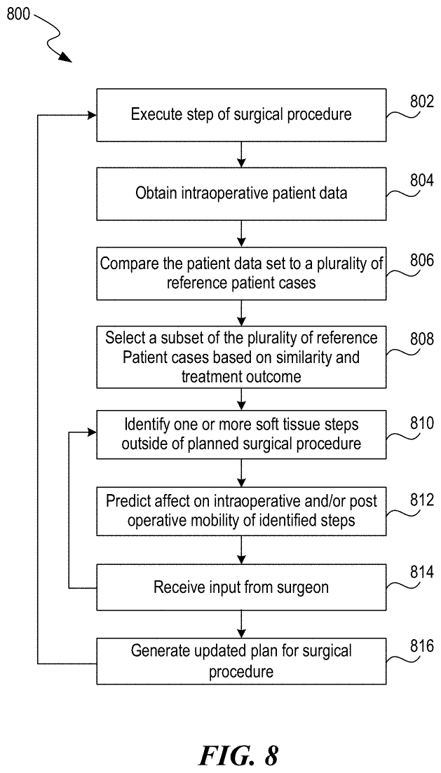

FIG. 8 is a flow diagram illustrating an intraoperative method 800 (“method 800”) for adapting a patient-specific plan for a surgical procedure to patient-specific anatomical structures during the surgical procedure in accordance with further embodiments of the present technology. The method begins at block 802 with executing one or more steps of a surgical procedure. The step can be executed by a surgeon, other healthcare provider, and/or a surgical robot executing portions of (or all of) a plan for a surgical procedure.

At block 804, the method 800 includes obtaining intraoperative patient data. As discussed above, the interoperative patient data can include intraoperative image data, a measurement of the anatomical response to the executed step of the surgical procedure, an update to the virtual model of the patient, and/or any other suitable type of intraoperative data.

At block 806, the method 800 includes comparing the updated patient data to a plurality of reference patient cases. Like the processes and server modules discussed in more detail above with respect to FIGS. 1 and 5A-5C, the method 800 can compare the updated patient data to the reference patient cases to identify similar data and/or similar cases. The comparison can be based on any of the data parameters discussed above with respect to FIGS. 1-7. The parameter(s) can be used to calculate a similarity score for each reference patient case, where the similarity score can represent a statistical correlation between the patient data and the reference patient case.

At block 808, the method 800 includes selecting a subset of the plurality of reference patient cases. The subset of reference patient cases can be selected based on a similarity between the patient data and the subset of reference patient cases and/or treatment outcomes in the subset of reference patient cases. For example, the subset of reference patient cases can be selected based on the similarity score generated at block 806 and desired treatment outcomes in the reference patient cases to identify both (a) patients with similar anatomical and/or biological features and (b) treatments that resulted in favorable outcomes for the similar patients.

At block 810, the method 800 includes identifying one or more additional steps for the plan for the surgical procedure. In some embodiments, the one or more additional steps can be identified based on additional steps taken in the similar patients with desired treatment outcomes. In some embodiments, the identification can include training a machine learning model, AI model, neural network, or the like on the subset of the plurality of reference patient cases. As discussed in more detail above, the trained machine learning model, AI model, neural network, and/or similar computer model can then be applied to the patient data to identify one or more additional steps. It is believed that the trained models can identify additional steps that improve the treatment outcomes achieved before healthcare providers recognize causal mechanisms between the additional steps and the improved treatment outcomes. It is also believed that the trained models can identify limitations in the additional steps to patient-specific features with improved accuracy over known limitations.

At block 812, the method 800 includes predicting an outcome of the surgical procedure if one or more of the additional steps identified at block 810 are incorporated into the plan for the surgical procedure. As discussed above, in various embodiments, the method 800 can include predicting the outcome for surgical procedures including each of the additional steps individually and/or each possible combination of the additional steps. In some embodiments, predicting the outcome includes training a machine learning model, an AI model, neural network, or the like to predict the anatomical configuration resulting from the surgical procedures that include one or more of the additional steps. For example, as discussed above, reference patient cases can be used to train a machine learning model to predict the anatomical effect and/or outcomes associated with one or surgical steps, sometimes isolated to reference patients that have similarities with the subject patient. In some such embodiments, predicting the outcome includes adjusting a virtual model of the patient to reflect the predicted anatomical configuration, allowing the healthcare provider (e.g., the surgeon) to visualize the predicted outcome. In some embodiments, predicting the outcome includes predicting whether surgical procedures that include one or more of the additional steps are likely to achieve a desired treatment outcome. In some embodiments, predicting the outcome includes generating an outcome score. The outcome score can reflect how closely a predicted treatment outcome is to the desired treatment outcome, whether the predicted treatment outcome is improved by the one or more additional steps, a risk factor associated with the one or more additional steps, and/or various other suitable evaluations of the predicted treatment outcome.

At block 814, the method 800 includes receiving an input from a surgeon (or other healthcare provider) regarding the additional steps identified at block 810. The input can include an identification of risks associated with the additional steps that the method 800 did not recognize, modifications to one or more of the additional steps, one or more additional steps the method 800 did not identify, a selection of one or more additional steps, an order for the one or more selected additional steps, and/or any other suitable indication.

Optionally, after receiving the input from the surgeon, the method 800 can return to block 810 to identify one or more further additional steps. For example, in embodiments in which a surgeon inputs a patient-specific risk, the method 800 can return to block 810 to identify further additional steps based on the patient-specific risk. The method 800 can then return to block 812 to predict an effect on the intraoperative and/or post-operative mobility of the anatomical feature being treated based on the further additional steps identified.

At block 816, the method 800 includes generating an updated plan for the surgical procedure based on the inputs from the surgeon and the additional steps identified and/or selected. As discussed above, the updated plan can be formatted for instruction to the surgeon and/or any other healthcare provider to follow, and/or for execution (partial or entire) by a surgical robot and/or any other suitable automated system. Further, as discussed above, generating the updated plan for the surgical procedure can include one or more steps to custom-manufacture an implant to account for the updated plan for the surgical procedure.

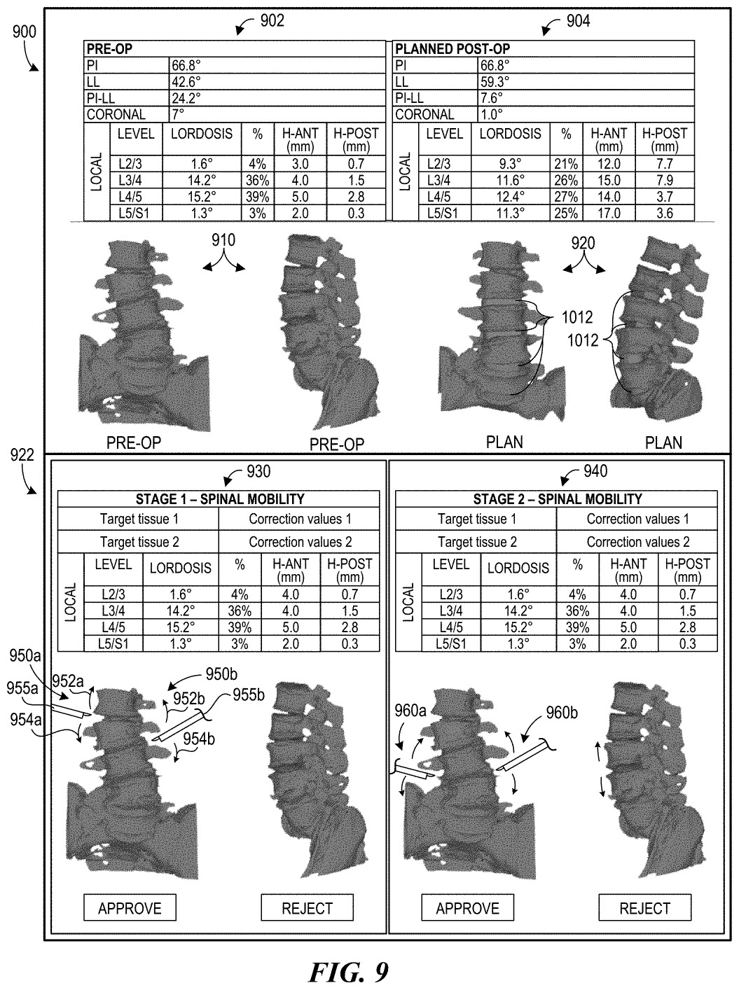

FIG. 9 illustrates an exemplary surgical plan 900 for a patient-specific surgical procedure that may be used and/or generated in connection with the methods described herein, according to an embodiment. The surgical plan 900 can incorporate all or some of surgical steps, analytics, and/or other data disclosed herein. For example, the surgical plan 900 can include output and parameters discussed in connection with methods of FIGS. 6-7. The surgical plan 900 can include, without limitation, intra- and/or pre-operative patient metrics 902 (e.g., pre-operative patient metrics discussed in connection with FIGS. 5A-5C), predicted post-operative patient metrics 904 (e.g., predicted post-operative patient metrics discussed in connection with FIGS. 5A-5C), targeted tissue for spinal mobility (e.g., tissue discussed in connection with FIG. 4C), correction values, simulation output, etc. The pre-operative information 902 can include views of anatomical features, such as anterior and lateral views of a virtual model 910 showing a native anatomical configuration of a patient. The anterior view of the virtual model 910 illustrates the patient has abnormal curvature (e.g., scoliosis) of his/her spinal column. The lateral view of the illustrated virtual model 910 shows the patient has collapsed discs or decreased spacing between adjacent vertebral endplates. The planned post-operative data 904 can include views of anatomical features, such as anterior and lateral views of a planned post-operative corrected virtual 920 showing the corrected anatomical configuration (including vertebral repositioning 1012) for the same patient. The virtual model 920 accounts for the abnormal anatomical configurations shown in the pre-operative model 910. A user can visually compare the pre- and post-operative data 902, 904 to evaluate predicted surgical outcomes.

The surgical plan 900 can also include an intraoperative plan or data 922 (“intraoperative plan 922”). The intraoperative plan 922 can include data (e.g., surgical steps, spinal mobility, etc.) for one or more stages of the surgical procedure. The illustrated two-stage surgical plan 900 includes first stage metrics 930 and second stage metrics 940, which can include any number of soft tissue surgical steps to adjust intraoperative mobility and can include data (e.g., soft tissue data, ancillary steps/procedures data, predicted outcomes, updated intraoperative plans, obstacles to surgical steps, predicted effects on intra-operative mobility, and/or post-operative mobility, etc.) discussed in connection with, for example, FIGS. 4C, 6, 7, and 8. A user can select a type of surgical procedure, stage parameter(s) (e.g., maximum or minimum number of stages), surgical step parameters (e.g., select surgical steps per stage, per surgical procedure, etc.), and other surgical plan parameters. In some embodiments, multiple intraoperative plans can be generated and displayed for visual comparison by the user. The user can provide input (e.g., approval, rejection, modification, etc.) for one or more portions of the surgical plan. The system can generate any number of plans (e.g., plans 900, 922) until receiving user approval.

With continued reference to FIG. 9, the first stage 930 can include planned soft tissue surgical steps 950a, 950b (collectively “surgical steps 950”) simulated using virtual models (e.g., virtual models discussed in connection with FIGS. 6, 7, and 8). The first stage 930 can include severing/cutting targeted tissue (e.g., targeted ligaments, connective tissue, etc.) and associated metrics (e.g., intraoperative metrics, postoperative metrics, correction values, spine metrics, etc.) reviewable by the physician. The virtual models can help the physician plan intraoperative surgical steps by, for example, reordering, adding, eliminating, and/or modifying surgical steps. The preoperative data 902, post-operative data 904, and/or intraoperative plan 922 can be updated based on the physician input.

The physician can review and approve the first stage metrics 930 by selecting an approve button. The computing system can then design mobility-adjustment surgical steps, instruments, implants, etc., based on the approved steps 950 (e.g., design intraoperative mobility of vertebrae of the spine to achieve the corrected anatomical configuration for approved adjustment(s)/outcome(s)). If the physician wants to modify intraoperative mobility, the physician can select the modify button. The physician can then input one or more parameters or metrics for adjustment. Examples of physician input are discussed in connection with, for example, blocks 606 and 614 of FIG. 6, block 712 of FIG. 7, and block 814 of FIG. 8. The computing system can update the spinal model accordingly to the inputted parameters, metrics, or other input.

The virtual models of the first stage include arrows (e.g., arrows 952a, 954a) indicating intraoperative mobility (e.g., range of motion, degrees of freedom, predicted correction values, location of predicted correction values, etc.) associated with an instrument 955a alternating first targeted tissue (e.g., cutting intertransverse ligaments on the patient's right side). The correction values can include at least one of a maximum distraction, lordosis correction, kyphosis correction, scoliosis correction, and/or spondylolisthesis correction. Arrows (e.g., arrows 952b, 954b) indicate intraoperative mobility (e.g., range of motion, degrees of freedom, predicted correction values, location of predicted correction values, etc.) associated with an instrument 955b alternating soft tissue (e.g., cutting intertransverse ligaments on the patient's left side). A user can modify or approve the adjust ability of the implant, based on the arrows.

The second stage metrics 940 can include intraoperative mobility associated with surgical steps 960a, 960b at different levels (illustrated as lower levels of the spine) indicating intraoperative mobility associated with steps performed using the instruments 960a, 960b. A user can modify or approve the adjust ability of the spinal mobility based on the arrows, predicted metrics, etc. For fusion procedures, the system can simulate one or more types of spinal fusion procedures (e.g., LIF, ALIF, PLIF, TLIF, etc.) by selecting access paths, tissue to be cut (e.g., anterior longitudinal ligament for ALIF, intertransverse ligament for TLIF, etc.) and virtually perform the procedure(s) using one or more virtual models. The simulations can be generated using three-dimensional models, surfaces, and/or virtual representations. The simulations can be generated using, for example, CAD software, finite element analysis (FEA) software (e.g., analyze stress in implants, tissue, etc.), or the like based on patient data, instrument configurations, implant designs data, or the like. A user can view, manipulate (e.g., rotate, move, etc.), modify, set parameters (e.g., boundary conditions, properties, etc.), and/or interact with the models.

The physician can approve/select individual target intraoperative configurations and/or post-operative configurations for different loading conditions. The surgical steps in a stage, number of stages, generated metrics, virtual models, and other data can be selected based on the surgical procedure, user input, etc. The plans for adjusting mobility can be generated using one or more machine learning algorithms. The machine learning algorithms can be based, at least partially, on reference patient data sets. The reference patient data sets can include data on a reference patient's spine, a reference surgical procedure performed on the reference patient's spine, adjustable mobility, intraoperative data, and/or an outcome of the reference surgical procedure. The machine learning algorithms can be updated (e.g., retrained) using new or updated patient data to determine adjustments to the plan for the surgical procedure. Other training techniques can be used to generate plans, stages, etc. In some embodiments, pre-operative patient data, intraoperative patient data, or both can be used to generate plans. For example, intraoperative patient data can be inputted into machine learning algorithms to generate plans during surgical procedures.

It will be understood that any of the systems and methods discussed above with respect to FIGS. 1-9 can be executed multiple times for a single patient, including in multiple times for a single medical treatment plan. For example, in embodiments in which the systems and methods are applied to a corrective spinal surgical procedure, the systems and methods described above can be applied on a per level scale, a per two-level scale, a per three-level scale, a per region scale, or any other suitable scale in addition to the patient's spine altogether. For example, the plan generation methods can be executed repetitively to generate a plurality of surgical procedure plans that are each specific to a vertebral pair, then executed to compile a complete plan for the surgical procedure based on the plurality of surgical procedure plans. In such embodiments, the iterations between vertebral pairs helps identify small additional steps specific to each vertebral pair that will improve the overall surgical procedure, while the iterations compiling the plurality of plans help identify larger-scale additional steps and/or realize efficiencies between the plurality of plans.

Further, in any of the systems and methods discussed above with respect to FIGS. 1-9, patient data can also be collected periodically after the medical treatment. The post-treatment patient data can then be used to quantify an outcome of the medical treatment, such as the anatomical correction actually provided by a surgical procedure and/or an evaluation of the success of the surgical procedure. For example, the post-treatment patient data can include image data of the patient's anatomical features; an assessment of the soft tissue features surrounding the anatomical features; an assessment from the patient regarding their pain, experience, mobility, and/or flexibility; and/or any other suitable point of data. The post-treatment patient data can then be used as a reference case for further medical treatments using the systems and methods disclosed herein. For example, the post-treatment patient data can be used to train further machine learning models, AI models, neural networks, and the like for a new patient. Accordingly, by collecting the post-treatment patient data, the system can identify which additional steps actually resulted in improved outcomes, which additional steps most-improved outcomes, which steps did not result in improved outcomes, what patient-specific features may lead to improved outcomes, and the like. Over time, the systems and methods disclosed herein are therefore expected to increase the efficacy of medical treatment.

### EXAMPLES

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples can be combined in any suitable manner, and placed into a respective independent example. The other examples can be presented in a similar manner.

1. A computer-implemented method for modeling a surgical correction for a patient, the method comprising:
obtaining patient data including image data of one or more regions of a patient's spine, wherein the image data depicts a native anatomical configuration of the patient's spine;
generating a virtual model of the patient's spine in a corrected anatomical configuration;
identifying one or more soft tissue surgical steps for adjusting intraoperative mobility of vertebrae of the spine to achieve the corrected anatomical configuration; and
generating a surgical plan for achieving the corrected anatomical configuration, wherein the surgical plan includes at least one of the soft tissue surgical steps that facilitates movement of the vertebrae to the corrected anatomical configuration.

2. The computer-implemented method of example 1, further comprising:
simulating the intraoperative mobility using the virtual model for viewing by a user; and identifying intraoperative mobility in the simulation attributable to the one or more soft tissue surgical steps.

3. The computer-implemented method of example 2, further comprising:
receiving user selection of at least one of the soft tissue surgical steps; and
updating the simulation to represent intraoperative mobility attributable to the selected soft tissue surgical steps.

4. The computer-implemented method of any of examples 1-3, further comprising:
generating surgical steps for the surgical plan; and
virtually simulating the surgical steps for viewing by a physician.

5. The computer-implemented method of any of examples 1-4, further comprising:
predicting intraoperative spinal mobility based on the one or more soft tissue surgical steps being performed; and
determining one or more intraoperative correction values based on the predicted mobility, wherein the intraoperative correction values includes at least one of a maximum distraction, lordosis correction, kyphosis correction, scoliosis correction, and spondylolisthesis correction.

6. The computer-implemented method of any of examples 1-5, further comprising:
predicting post-operative spinal mobility based on the one or more soft tissue surgical steps being performed.

7. The computer-implemented method of any of examples 1-6, further comprising:
generating an intraoperative simulation of the adjusted intraoperative mobility of vertebrae of the spine attributable to the identifying one or more soft tissue surgical steps,
providing a physician viewing of the intraoperative simulation, and
receiving input, from the physician, wherein the input in the generating the surgical plan.

8. The computer-implemented method of any of examples 1-7, further comprising:
receiving physician input for an ancillary surgical procedure, wherein the one or more soft tissue surgical steps are selected based on the receiving the physician input, wherein the ancillary surgical procedure is part of the surgical plan.

9. The computer-implemented method of any of examples 1-8, further comprising:
simulating joint mobility of the patient's spine;
selecting one or more of the identified soft tissue surgical steps based on the simulated joint mobility; and
predicting post-operative joint mobility associated with the selected one or more soft tissue surgical steps.

10. The computer-implemented method of any of examples 1-9, wherein the one or more soft tissue surgical steps includes:
severing a ligament along the patient's spine,
removing at least a portion of an annulus of intervertebral disc, and
resecting cartilage along the spine.

11. The computer-implemented method of any of examples 1-10, wherein the one or more soft tissue surgical steps includes a decompression procedure.

12. The computer-implemented method of example 11, further comprising predicting a nerve decompression score for the decompression procedure.

13. The computer-implemented method of any of examples 11 and 12, further comprising:
generating a plurality of decompression plans;
determining a decompression score for each decompression plan;
receiving selection of one of the decompression plans; and
generating a decompression surgical plan based on the selected decompression plan.

14. The computer-implemented method of example 13, wherein the decompression plans includes at least one of a laminectomy, a laminotomy, a microdiscectomy, a foraminotomy, or an osteophyte procedure.

15. A computer-implemented method for modeling a surgical correction, the method comprising:
obtaining patient data including image data of one or more regions of a patient's spine, wherein the image data depicts a native anatomical configuration of the patient's spine;
generating at least one corrected anatomical configuration for the patient's spine;
receiving input on a primary spine procedure to adjust the patient's spine towards the corrected anatomical configuration;
identifying a set of ancillary spine procedures;
receiving selection of one of the ancillary spine procedures; and predicting an outcome for the selected ancillary spine procedure based on the patient's spine in the corrected anatomical configuration.

16. The method of example 15, wherein the set of ancillary spine procedures includes:
a laminectomy,
a laminotomy,
a microdiscectomy,
a foraminotomy, and
an osteophyte procedure.

17. The method of any of examples 15 and 16, further comprising virtually simulating the selected one of the ancillary spine procedures.

18. A computer-implemented method for modeling a surgical correction to an anatomical configuration, the method comprising:
obtaining patient data, the patient data including image data of one or more regions of a patient's spine, wherein the image data depicts a native anatomical configuration;
generating a virtual model of the patient's spine in a based on the native anatomical configuration;
determining a target anatomical configuration for the one or more regions, wherein the target anatomical configuration is different than the native anatomical configuration;
identifying one or more intraoperative surgical alterations to soft tissue features surrounding the patient's spine to adjust an interoperative mobility of the patient's spine, wherein the one or more intraoperative surgical alterations to the soft tissue features are identified based at least in part on the virtual model of the patient's spine and the target anatomical configuration; and
predicting a resulting anatomical configuration based at least in part on the one or more intraoperative surgical alterations to soft tissue features surrounding the patient's spine and the patient data.

19. The computer-implemented method of example 18 wherein identifying one or more intraoperative surgical alterations to soft tissue features surrounding the patient's spine includes:
training a machine learning algorithm based at least partially on a plurality of reference patient data sets, each of the plurality of reference patient data sets having data on a reference patient's spine, a reference intraoperative surgical alterations performed on the reference patient's spine, and a reference resulting anatomical configuration; and
applying the machine learning algorithm to the virtual model of the patient's spine and the target anatomical configuration.

20. The computer-implemented method of any of examples 18 and 19 wherein predicting the resulting anatomical configuration includes:
training a machine learning algorithm based at least partially on a plurality of reference patient data sets and the one or more intraoperative surgical alterations, wherein each of the plurality of reference patient data sets having data on a reference patient's spine, a reference intraoperative surgical alterations performed on the reference patient's spine, and a reference resulting anatomical configuration; and
applying the machine learning algorithm to the virtual model of the patient's spine and the one or more intraoperative surgical alterations.

21. The computer-implemented method of any of examples 18-20, further comprising:
receiving, from a surgeon, one or more inputs comprising adjustments to the one or more intraoperative surgical alterations; and
updating the prediction of the resulting anatomical configuration based on the inputs from the surgeon.

22. The computer-implemented method of any of examples 18-21, further comprising generating instructions for performing the one or more intraoperative surgical alterations.

23. The computer-implemented method of any of examples 18-24 wherein predicting the resulting anatomical configuration includes identifying obstacles to completing the one or more intraoperative surgical alterations.

24. The computer-implemented method of any of examples 18-23 wherein predicting the resulting anatomical configuration includes identifying risks associated with the one or more intraoperative surgical alterations.

25. A computer-implemented method for modeling an orthopedic correction, the method comprising:
generating a virtual model of a patient's spine, the virtual model representing an anatomical configuration of at least a portion of a patient's spine and one or more soft tissue features surrounding the portion of the patient's spine;
determining a desired anatomical configuration for the portion of the patient's spine;
receiving an indication of a surgical procedure plan based at least in part on the desired anatomical configuration; and
comparing the virtual model of the patient's spine and the surgical procedure plan to a plurality of reference cases;
selecting a subset of the plurality of reference cases based at least in part on a similarity between the virtual model of the patient's spine and the surgical procedure plan and the subset of the plurality of reference cases;
predicting an outcome of the surgical procedure plan based at least in part on the subset of the plurality of reference cases.

26. The computer-implemented method of example 25, further comprising:
identifying one or more alterations to the surgical procedure plan, each of the one or more alterations including one or more steps for manipulating the portion of the patient's spine to adjust intraoperative mobility of the portion of the patient's spine, and
wherein predicting the outcome of the surgical procedure plan is further based at least in part on the one or more alterations to the surgical procedure plan.

27. The computer-implemented method of example 26 wherein identifying the one or more alterations to the surgical procedure plan includes:
training a machine learning algorithm based at least partially on the subset of the plurality of reference cases; and
applying the machine learning algorithm to the virtual model of the patient's spine and the surgical procedure plan.

28. The computer-implemented method of any of examples 26 and 27, wherein the one or more alterations to the surgical procedure plan address patient-specific obstacles, and wherein the patient-specific obstacles include one or more of: a patient-specific feature of a vertebral body of in the patient's spine, a patient specific soft tissue feature surrounding the patient's spine, a patient-specific anomaly in bone density, a trend in bone recovery in the subset of the plurality of reference cases, and a trend in soft tissue recovery in the subset of the plurality of reference cases.

29. The computer-implemented method of example 28 wherein identifying the one or more alterations to the surgical procedure plan includes predicting an intraoperative mobility of the patient's spine, and wherein the patient-specific obstacles include patient-specific features that effect the intraoperative mobility of the patient's spine.

30. The computer-implemented method of any of examples 26-29 wherein identifying one or more alterations to the surgical procedure plan includes identifying candidate locations to severe a spinal ligament.

31. The computer-implemented method of any of examples 26-30 wherein predicting the outcome of the surgical procedure plan includes:
training a machine learning algorithm based at least partially on the subset of the plurality of reference cases; and
applying the machine learning algorithm to the virtual model of the patient's spine and the one or more alterations to the surgical procedure plan.

32. The computer-implemented method of any of examples 25-31 wherein predicting the outcome of the surgical procedure plan includes predicting a post-operative mobility of the patient's spine.

33. The computer-implemented method of any of examples 25-32, further comprising:
obtaining surgery technique data specific to the patient and a surgeon, the surgery technique data indicating one or more of: a preferred operation technique for the surgeon, a preferred operation technique for the patient, and a record of outcomes specific to the surgeon,
wherein predicting the outcome of the surgical procedure plan is further based on the surgery technique data.

34. A computer-implemented method for intraoperatively adjusting a surgical procedure, the method comprising:
obtaining a patient data and a plan for the surgical procedure, the patient data including a virtual model of at least a portion of a patient's spine and one or more soft tissue features surrounding the portion of the patient's spine;
receiving, from a surgeon, one or more updates to the patient data, the updates including intraoperative data on the portion of the patient's spine and/or the one or more soft tissue features surrounding the portion of the patient's spine;
generating, based at least partially on the updated patient data, one or more options for adjustments to the plan for the surgical procedure; and
predicting, for each of the one or more options adjustments to the plan for the surgical procedure, an outcome of the surgical procedure based on the adjustments to the plan and the updated patient data.

35. The computer-implemented method of example 34 wherein generating the one or more options for adjustments to the plan includes:
training a machine learning algorithm based at least partially on a plurality of reference patient data sets, each of the plurality of reference patient data sets having data on a reference patient's spine, a reference surgical procedure performed on the reference patient's spine, and an outcome of the reference surgical procedure; and
applying the machine learning algorithm to the updated patient data to determine the one or more adjustments to the plan for the surgical procedure.

36. The computer-implemented method of any of examples 34 and 35 wherein predicting the outcome of the surgical procedure for each of the one or more options for adjustments to the plan includes:
training a machine learning algorithm based at least partially on a plurality of reference patient data sets, each of the plurality of reference patient data sets having data on a reference patient's spine, a reference surgical procedure performed on the reference patient's spine, and an outcome of the reference surgical procedure; and
applying the machine learning algorithm to each of the one or more options for adjustments to the plan and the updated patient data.

Those skilled in the art will recognize that it is common within the art to describe devices and/or processes in the fashion set forth herein, and thereafter use engineering practices to integrate such described devices and/or processes into data processing systems for performing the computer-implemented methods. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

The embodiments, examples, features, systems, devices, materials, methods and techniques described herein may, in some embodiments, be similar to any one or more of the embodiments, examples, features, systems, devices, materials, methods and techniques described in the following:

U.S. application Ser. No. 16/048,167, filed on Jul. 27, 2017, titled “SYSTEMS AND METHODS FOR ASSISTING AND AUGMENTING SURGICAL PROCEDURES;”

U.S. application Ser. No. 16/242,877, filed on Jan. 8, 2019, titled “SYSTEMS AND METHODS OF ASSISTING A SURGEON WITH SCREW PLACEMENT DURING SPINAL SURGERY;”

U.S. application Ser. No. 16/207,116, filed on Dec. 1, 2018, titled “SYSTEMS AND METHODS FOR MULTI-PLANAR ORTHOPEDIC ALIGNMENT;”

U.S. application Ser. No. 16/352,699, filed on Mar. 13, 2019, titled “SYSTEMS AND METHODS FOR ORTHOPEDIC IMPLANT FIXATION;”

U.S. application Ser. No. 16/383,215, filed on Apr. 12, 2019, titled “SYSTEMS AND METHODS FOR ORTHOPEDIC IMPLANT FIXATION;”

U.S. application Ser. No. 16/569,494, filed on Sep. 12, 2019, titled “SYSTEMS AND METHODS FOR ORTHOPEDIC IMPLANTS;”

U.S. Application No. 62/773,127, filed on Nov. 29, 2018, titled “SYSTEMS AND METHODS FOR ORTHOPEDIC IMPLANTS;”

U.S. Application No. 62/928,909, filed on Oct. 31, 2019, titled “SYSTEMS AND METHODS FOR DESIGNING ORTHOPEDIC IMPLANTS BASED ON TISSUE CHARACTERISTICS;”

U.S. application Ser. No. 16/735,222, filed Jan. 6, 2020, titled “PATIENT-SPECIFIC MEDICAL PROCEDURES AND DEVICES, AND ASSOCIATED SYSTEMS AND METHODS;”

U.S. application Ser. No. 16/987,113, filed Aug. 6, 2020, titled “PATIENT-SPECIFIC ARTIFICIAL DISCS, IMPLANTS AND ASSOCIATED SYSTEMS AND METHODS;”

U.S. application Ser. No. 16/990,810, filed Aug. 11, 2020, titled “LINKING PATIENT-SPECIFIC MEDICAL DEVICES WITH PATIENT-SPECIFIC DATA, AND ASSOCIATED SYSTEMS, DEVICES, AND METHODS;”

U.S. application Ser. No. 17/085,564, filed Oct. 30, 2020, titles “SYSTEMS AND METHODS FOR DESIGNING ORTHOPEDIC IMPLANTS BASED ON TISSUE CHARACTERISTICS;”

U.S. application Ser. No. 17/100,396, filed Nov. 20, 2020, titled “PATIENT-SPECIFIC VERTEBRAL IMPLANTS WITH POSITIONING FEATURES;” and

U.S. Provisional Patent Application No. 63/223,827, filed Jul. 20, 2021, titled “SYSTEMS FOR PREDICTING INTRAOPERATIVE PATIENT MOBILITY AND IDENTIFYING MOBILITY-RELATED SURGICAL STEPS.”

All of the above-identified patents and applications are incorporated by reference in their entireties. In addition, the embodiments, features, systems, devices, materials, methods and techniques described herein may, in certain embodiments, be applied to or used in connection with any one or more of the embodiments, features, systems, devices, or other matter.

### CONCLUSION

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any material incorporated herein by reference conflicts with the present disclosure, the present disclosure controls. Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising,” “including,” “having,” and “with” are used throughout to mean including at least the recited feature(s) such that any greater number of the same features and/or additional types of other features are not precluded.

From the foregoing, it will also be appreciated that various modifications may be made without deviating from the disclosure or the technology. For example, one of ordinary skill in the art will understand that various components of the technology can be further divided into subcomponents, or that various components and functions of the technology may be combined and integrated. In addition, certain aspects of the technology described in the context of particular embodiments may also be combined or eliminated in other embodiments. Furthermore, although advantages associated with certain embodiments of the technology have been described in the context of those embodiments, other embodiments may also exhibit such advantages, and not all embodiments need necessarily exhibit such advantages to fall within the scope of the technology.

Accordingly, the disclosure and associated technology can encompass other embodiments not expressly shown or described herein.

What is claimed is:

1. A computer-implemented method for modeling a surgical correction for a patient, the method comprising:
obtaining patient data including image data of one or more regions of a patient's spine, wherein the image data depicts a native anatomical configuration of the patient's spine;
generating a virtual model of the patient's spine in a corrected anatomical configuration;
identifying one or more soft tissue surgical steps for adjusting intraoperative mobility of vertebrae of the spine to achieve the corrected anatomical configuration; and
generating a surgical plan for achieving the corrected anatomical configuration, wherein the surgical plan includes at least one of the soft tissue surgical steps that facilitates movement of the vertebrae to the corrected anatomical configuration.

2. The computer-implemented method of claim 1, further comprising:
simulating the intraoperative mobility using the virtual model for viewing by a user; and
identifying intraoperative mobility in the simulation attributable to the one or more soft tissue surgical steps.

3. The computer-implemented method of claim 2, further comprising:
receiving user selection of at least one of the soft tissue surgical steps; and
updating the simulation to represent intraoperative mobility attributable to the selected soft tissue surgical steps.

4. The computer-implemented method of claim 1, further comprising:
generating surgical steps for the surgical plan; and
virtually simulating the surgical steps for viewing by a physician.

5. The computer-implemented method of claim 1, further comprising:
predicting intraoperative spinal mobility based on the one or more soft tissue surgical steps being performed; and
determining one or more intraoperative correction values based on the predicted mobility, wherein the intraoperative correction values includes at least one of a maximum distraction, lordosis correction, kyphosis correction, scoliosis correction, or spondylolisthesis correction.

6. The computer-implemented method of claim 1, further comprising:
predicting post-operative spinal mobility based on the one or more soft tissue surgical steps being performed.

7. The computer-implemented method of claim 1, further comprising:
generating an intraoperative simulation of the adjusted intraoperative mobility of vertebrae of the spine attributable to the identifying one or more soft tissue surgical steps,
providing a physician viewing of the intraoperative simulation, and
receiving input, from the physician, wherein the input in the generating the surgical plan.

8. The computer-implemented method of claim 1, further comprising:
receiving physician input for an ancillary surgical procedure, wherein the one or more soft tissue surgical steps are selected based on the receiving the physician input, wherein the ancillary surgical procedure is part of the surgical plan.

9. The computer-implemented method of claim 1, further comprising:
simulating a joint mobility of the patient's spine;
selecting one or more of the identified soft tissue surgical steps based on the simulated joint mobility; and
predicting post-operative joint mobility associated with the selected one or more soft tissue surgical steps.

10. The computer-implemented method of claim 1, wherein the one or more soft tissue surgical steps includes:
severing a ligament along the patient's spine,
removing at least a portion of an annulus of intervertebral disc, and
resecting cartilage along the spine.

11. The computer-implemented method of claim 1, wherein the one or more soft tissue surgical steps includes a decompression procedure.

12. The computer-implemented method of claim 11, further comprising predicting a nerve decompression score for the decompression procedure.

13. The computer-implemented method of claim 1, further comprising:
generating a plurality of decompression plans;
determining a decompression score for each decompression plan;
receiving selection of one of the decompression plans; and
generating a decompression surgical plan based on the selected decompression plan.

14. The computer-implemented method of claim 13, further comprising identifying one or more bony tissues for removal to adjust the intraoperative mobility, wherein the decompression plans include at least one of a laminectomy, a laminotomy, a microdiscectomy, a foraminotomy, and an osteophyte procedure.

15. A computer-implemented method for modeling a surgical correction, the method comprising:
obtaining patient data including image data of one or more regions of a patient's spine, wherein the image data depicts a native anatomical configuration of the patient's spine;
generating at least one corrected anatomical configuration for the patient's spine;
receiving input on a primary spine procedure to adjust the patient's spine towards the corrected anatomical configuration;
identifying a set of ancillary spine procedures;
receiving selection of one of the ancillary spine procedures; and
predicting an outcome for the selected ancillary spine procedure based on the patient's spine in the corrected anatomical configuration.

16. The method of claim 15, wherein the set of ancillary spine procedures includes:
a laminectomy,
a laminotomy,
a microdiscectomy,
a foraminotomy, and
an osteophyte procedure.

17. The method of claim 15, further comprising virtually simulating the selected one of the ancillary spine procedures.

18. A computer-implemented method for modeling a surgical correction to an anatomical configuration, the method comprising:
obtaining patient data, the patient data including image data of one or more regions of a patient's spine, wherein the image data depicts a native anatomical configuration;
generating a virtual model of the patient's spine in a based on the native anatomical configuration;
determining a target anatomical configuration for the one or more regions, wherein the target anatomical configuration is different than the native anatomical configuration;
identifying one or more intraoperative surgical alterations to soft tissue features surrounding the patient's spine to adjust an interoperative mobility of the patient's spine, wherein the one or more intraoperative surgical alterations to the soft tissue features are identified based at least in part on the virtual model of the patient's spine and the target anatomical configuration; and
predicting a resulting anatomical configuration based at least in part on the one or more intraoperative surgical alterations to the soft tissue features surrounding the patient's spine and the patient data.

19. The computer-implemented method of claim 18 wherein identifying one or more intraoperative surgical alterations to soft tissue features surrounding the patient's spine includes:
training a machine learning algorithm based at least partially on a plurality of reference patient data sets, each of the plurality of reference patient data sets having data on a reference patient's spine, a reference intraoperative surgical alterations performed on the reference patient's spine, and a reference resulting anatomical configuration; and
applying the machine learning algorithm to the virtual model of the patient's spine and the target anatomical configuration.

20. The computer-implemented method of claim 18 wherein predicting the resulting anatomical configuration includes:
training a machine learning algorithm based at least partially on a plurality of reference patient data sets and the one or more intraoperative surgical alterations, wherein each of the plurality of reference patient data sets having data on a reference patient's spine, a reference intraoperative surgical alterations performed on the reference patient's spine, and a reference resulting anatomical configuration; and
applying the machine learning algorithm to the virtual model of the patient's spine and the one or more intraoperative surgical alterations.

21. The computer-implemented method of claim 18, further comprising:
receiving, from a surgeon, one or more inputs comprising adjustments to the one or more intraoperative surgical alterations; and
updating the prediction of the resulting anatomical configuration based on the inputs from the surgeon.

22. The computer-implemented method of claim 18, further comprising generating instructions for performing the one or more intraoperative surgical alterations.

23. The computer-implemented method of claim 18 wherein predicting the resulting anatomical configuration includes identifying obstacles to completing the one or more intraoperative surgical alterations.

24. The computer-implemented method of claim 18 wherein predicting the resulting anatomical configuration includes identifying risks associated with the one or more intraoperative surgical alterations to the soft tissue features.

25. A computer-implemented method for modeling an orthopedic correction, the method comprising:
generating a virtual model of a patient's spine, the virtual model representing an anatomical configuration of at least a portion of a patient's spine and one or more soft tissue features surrounding the portion of the patient's spine;
determining a desired anatomical configuration for the portion of the patient's spine;
receiving an indication of a surgical procedure plan based at least in part on the desired anatomical configuration; and
comparing the virtual model of the patient's spine and the surgical procedure plan to a plurality of reference cases;
selecting a subset of the plurality of reference cases based at least in part on a similarity between the virtual model of the patient's spine and the surgical procedure plan and the subset of the plurality of reference cases;
predicting an outcome of the surgical procedure plan based at least in part on the subset of the plurality of reference cases.

26. The computer-implemented method of claim 25, further comprising:
identifying one or more alterations to the surgical procedure plan, each of the one or more alterations including one or more steps for manipulating the portion of the patient's spine to adjust intraoperative mobility of the portion of the patient's spine, and
wherein predicting the outcome of the surgical procedure plan is further based at least in part on the one or more alterations to the surgical procedure plan.

27. The computer-implemented method of claim 26 wherein generating the one or more alterations to the surgical procedure plan includes:
training a machine learning algorithm based at least partially on the subset of the plurality of reference cases; and
applying the machine learning algorithm to the virtual model of the patient's spine and the surgical procedure plan.

28. The computer-implemented method of claim 26, wherein the one or more alterations to the surgical procedure plan address patient-specific obstacles, and wherein the patient-specific obstacles include one or more of: a patient-specific feature of a vertebral body of in the patient's spine, a patient specific soft tissue feature surrounding the patient's spine, a patient-specific anomaly in bone density, a trend in bone recovery in the subset of the plurality of reference cases, and a trend in soft tissue recovery in the subset of the plurality of reference cases.

29. The computer-implemented method of claim 28 wherein generating the one or more alterations to the surgical procedure plan includes predicting an intraoperative mobility of the patient's spine, and wherein the patient-specific obstacles include patient-specific features that effect the intraoperative mobility of the patient's spine.

30. The computer-implemented method of claim 26 wherein generating one or more alterations to the surgical procedure plan includes identifying candidate locations to severe a spinal ligament.

31. The computer-implemented method of claim 26 wherein predicting the outcome of the surgical procedure plan includes:
training a machine learning algorithm based at least partially on the subset of the plurality of reference cases; and
applying the machine learning algorithm to the virtual model of the patient's spine and the one or more alterations to the surgical procedure plan.

32. The computer-implemented method of claim 25 wherein predicting the outcome of the surgical procedure plan includes predicting a post-operative mobility of the patient's spine.

33. The computer-implemented method of claim 25, further comprising:
obtaining surgery technique data specific to the patient and a surgeon, the surgery technique data indicating one or more of: a preferred operation technique for the surgeon, a preferred operation technique for the patient, and a record of outcomes specific to the surgeon,
wherein predicting the outcome of the surgical procedure plan is further based on the surgery technique data.

34. A computer-implemented method for intraoperatively adjusting a surgical procedure, the method comprising:
obtaining a patient data and a plan for the surgical procedure, the patient data including a virtual model of at least a portion of a patient's spine and one or more soft tissue features surrounding the portion of the patient's spine;
receiving, from a surgeon, one or more updates to the patient data, the updates including intraoperative data on the portion of the patient's spine and/or the one or more soft tissue features surrounding the portion of the patient's spine;
generating, based at least partially on the updated patient data, one or more options for adjustments to the plan for the surgical procedure; and
predicting, for each of the one or more options adjustments to the plan for the surgical procedure, an outcome of the surgical procedure based on the adjustments to the plan and the updated patient data.

35. The computer-implemented method of claim 34 wherein generating the one or more options for adjustments to the plan includes:
training a machine learning algorithm based at least partially on a plurality of reference patient data sets, each of the plurality of reference patient data sets having data on a reference patient's spine, a reference surgical procedure performed on the reference patient's spine, and an outcome of the reference surgical procedure; and
applying the machine learning algorithm to the updated patient data to determine the one or more adjustments to the plan for the surgical procedure.

36. The computer-implemented method of claim 34 wherein predicting the outcome of the surgical procedure for each of the one or more options for adjustments to the plan includes:
training a machine learning algorithm based at least partially on a plurality of reference patient data sets, each of the plurality of reference patient data sets having data on a reference patient's spine, a reference surgical procedure performed on the reference patient's spine, and an outcome of the reference surgical procedure; and
applying the machine learning algorithm to each of the one or more options for adjustments to the plan and the updated patient data.
